(19) United States
(12) Patent Application Publication
(10) Pub. No.: US 2009/0047689 A1
(43) Pub. Date: Feb. 19, 2009

(54) AUTOANTIGEN BIOMARKERS FOR EARLY DIAGNOSIS OF LUNG ADENOCARCINOMA

(76) Inventors: John Kolman, North Potomac, MD (US); Samir Hanash, Mercer Island, WA (US); Brad Love, Timonium, MD (US); Alexei Krasnoselsky, Shoreline, WA (US); Charles Kooperberg, Seattle, WA (US); Ji Qiu, Shoreline, WA (US)

Correspondence Address: GREENLEE WINNER AND SULLIVAN P C, 4875 PEARL EAST CIRCLE, SUITE 200, BOULDER, CO, 80301, US

(21) Appl. No.: 12/143,288

(22) Filed: Jun. 20, 2008

Related U.S. Application Data
(60) Provisional application No. 60/945,243, filed on Jun. 20, 2007.

(51) Int. Cl. G01N 33/574

(52) U.S. Cl. 435/7.23

(57) ABSTRACT

Provided herein are novel panels of biomarkers for the detection and diagnosis of lung adenocarcinoma, and methods and kits for detecting these biomarkers in samples of individuals suspected of having the disease. Also provided are methods of monitoring the progression of lung adenocarcinoma and methods of monitoring the efficacy of a treatment.

### CROSS REFERENCE TO RELATED APPLICATIONS

This application claims benefit of U.S. Provisional Application No. 60/945,243, filed Jun. 20, 2007, which is incorporated by reference in its entirety herein to the extent that there is no inconsistency with the present disclosure.

### ACKNOWLEDGMENT OF FEDERAL RESEARCH SUPPORT

Not Applicable.

### BACKGROUND OF THE INVENTION

This invention generally relates to biomarkers used to identify autoantibodies indicative of diseases, particularly lung cancer, and methods, compositions and kits for the diagnosis, prognosis, and monitoring the progression of such diseases.

The development of autoantibodies is observed in autoimmune disorders and numerous cancers. Because of this, proteins targeted by autoantibodies (herein referred to as “autoantigens”) are effective biomarkers and form the basis of potential diagnostic and prognostic assays, as well as approaches for monitoring disease progression and response to treatment. The effective use of autoantigen biomarkers for these applications, however, is often contingent upon the identification of not one but multiple biomarkers. This is a consequence of the observation that the development of autoantibodies to any given protein is typically seen only in a fraction of patients (A. Fossa et al., Prostate 59, 440-7 (Jun. 1, 2004); S. S. Van Rhee et al., Blood 105, 3939-3944 (2005)). Current methods for the identification of autoantigens are cumbersome, technically challenging, have low sensitivity, and poor reproducibility. It is therefore cumbersome and time-consuming to identify panels of disease-specific markers that could facilitate diagnosing and treating diseases.

One widely utilized approach for autoantigen identification is SEREX: serological analysis of cDNA expression libraries. This approach is most appropriate for cancer autoantigen identification, and involves the generation of tumor-specific lambda GT11 cDNA expression libraries, followed by immunological screening of plaque lifts using patient sera. The SEREX approach was successfully used to identify the cancer autoantigen NY-ESO-1, a protein that is autoantigenic in ˜20-50% of patients overexpressing NY-ESO-1 (Y. T. Chen et al., Proc Natl Acad Sci USA 94,1914-8 (1997)). However, while clearly useful, SEREX is not a high throughput approach, it is expensive, labor-intensive, requiring expertise in sophisticated molecular biological techniques, typically has a high false positive rate and, because it relies on bacterial protein expression, cannot identify autoantigens requiring post-translational modifications (U. Sahin et al., Proc Natl Acad Sci USA 92,11810-3 (1995)). More recently, reverse phase protein microarrays have been used to identify colon cancer and some lung cancer autoantigens (M. J. Nam et al., Proteomics 3, 2108-15 (2003); F. M. Brichory et al., Proc Natl Acad Sci USA 98, 9824-9 (2001)). These arrays are made by fractionating cancer cell homogenates, arraying them in spots on a microarray, probing them with patient sera, and detecting antibody binding. Mass-spectrometry based techniques are subsequently used to identify the actual autoantigen—a process which can be both time-consuming and tedious.

Functional protein microarrays are another method that may be used to identify biomarkers. These protein microarrays empower investigators with defined high-protein content for profiling serum samples to identify autoantigen biomarkers. Human protein microarrays may contain as many as 1800, 3000, 4000, 5000, 6000, 7000, 8000, 9000, 10,000, 100,000, 500,000 or 1,000,000 or more purified human proteins immobilized on nitrocellulose-coated glass slides. The protein microarrays may be probed with serum from a diseased individual to identify reactive proteins that are potential biomarkers for the disease. Human protein microarrays that contain proteins that are expressed in insect cells are expected to contain appropriate post-translational modifications. Because all proteins are purified under native conditions, immobilized proteins are expected to maintain their native conformations (B. Schweitzer, P. Predki, M. Snyder, Proteomics 3, 2190-9 (2003)).

Of particular interest, adenocarcinoma is a form of cancer originating in glandular tissue and forming glandular structures. Adenocarcinoma is common in the lung forming 30-40% of all lung carcinomas and typically arising from goblet cells or type II pneumocytes. The American Cancer Society estimates approximately 162,000 deaths per year due to lung cancer, with approximately 174,000 new cases of lung cancer arising each year. Detecting cancer early dramatically increases the chances of survival under existing treatment regimes. For example, colorectal, breast and prostrate cancer patients each have over a 90% 5-year survival rate following early detection compared to approximately 8%, 16%, 33% 5-year survival rate, respectively, after late detection. Lung cancer patients have an approximately 48% 5-year survival rate after early detection, if found before metastasis, compared to a less than 10% 5-year survival rate after late detection. Thus, it is tremendously important to be able to diagnose forms of cancer as soon as possible.

Current lung cancer diagnostics involve considering the patient's symptoms, such as persistent cough, deep and wheezing cough, bloody sputum, and difficulty breathing, in conjunction with results from tests, such as chest x-ray, CT scan, pulmonary function test and lung biopsy. What is needed is a method of diagnosing lung cancer that is simple to perform and minimally invasive. Although serum diagnostic tests are commonly used by physicians to confirm the presence of other diseases, no simple high-throughput diagnostic test is currently approved in the U.S. for testing serum or other fluids for lung cancer. There is a need in the art for the identification of new biomarkers that can be used in the care and management of lung cancer, particularly lung adenocarcinoma, by the development of a non-invasive, accurate, fast and sensitive assay that utilizes multiple biomarkers for the detection, diagnosis, staging, and monitoring of lung cancer in individuals.

The ability to screen a patient for multiple biomarkers associated with adenocarcinoma would improve diagnosis and treatment of the disease, and allow for earlier detection. However, it is unlikely that a single individual marker can accomplish this task. Assay experience with autoimmune diseases and cancer patients has demonstrated that a single antigen is not sufficient to characterize all sera and to differentiate between healthy and diseased individuals. An approach that can identify as many adenocarcinoma biomarkers as possible to generate a serological test will be beneficial so that patients can be selected for therapy based on accurate information regarding their antigenic profile.

### SUMMARY OF THE INVENTION

The present invention recognizes the need for a reliable and minimally invasive test for lung cancer, and in particular lung adenocarcinoma, that can be used as a supplement or replacement to current diagnostic methods. In one aspect, the invention provides an autoantigen array for use as a minimally invasive, multi-parametric screening test to detect and identify a form of cancer and its morphology. Furthermore, the present invention provides a screening method that can provide early detection of cancer prior to the development of a tumor mass or extensive cell division. The blood samples or other test samples from the patient are collected using routine means, allowing the screening method to be inexpensive and easy to use.

The invention is based in part on the discovery of a collection of autoantibody biomarkers for the detection, diagnosis, prognosis, staging, and monitoring of lung adenocarcinoma. The invention provides biomarkers for lung adenocarcinoma, particularly autoantibody biomarkers, and biomarker detection panels. Furthermore, the invention provides methods and kits detecting biomarkers for lung adenocarcinoma in a test sample of an individual.

The present invention identifies numerous biomarkers that are useful for the detection, diagnosis, staging, and monitoring of lung adenocarcinoma in individuals. A determination of the presence or absence of lung adenocarcinoma in an individual does not necessarily require that antibodies against all of the identified antigen biomarkers are present or absent. Similarly, a determination of the presence or absence of lung adenocarcinoma in an individual does not require that all of the target antigen biomarkers be present in increased or decreased amounts. Art-recognized statistical methods can be used to determine the significance of a specific pattern of antibodies against one or a plurality of the listed antigen biomarkers, or the significance of a specific pattern of increased or decreased amounts of biomarkers.

In one aspect of the invention, serum from patients diagnosed with lung adenocarcinoma as well as healthy patients were profiled against a human protein microarray containing thousands of human proteins (Table 1). Numerous proteins on the array were bound by antibodies from patients diagnosed with lung adenocarcinoma, but not healthy patients, identifying these proteins and the antibodies that recognize them as lung cancer biomarkers.

Additionally, serum was collected from patients having a high risk for lung cancer, such as smokers, former smokers, and workers exposed to asbestos, and profiled against a human protein microarray (Table 2). Some of these patients later developed lung adenocarcinoma. The screening results of test samples from patients one-year prior to being diagnosed with lung adenocarcinoma were compared with the screening results from healthy patients (patients one-year prior to being diagnosed with a disease free status). Numerous proteins on the array were bound by antibodies from patients later diagnosed with lung adenocarcinoma, but not healthy patients, identifying these proteins and the antibodies that recognize them as lung cancer biomarkers.

Numerous antigens associated with specific immunoglobulins have been also identified as being useful for the detection, diagnosis, staging, and monitoring of lung adenocarcinoma in individuals. Immunoglobulin G (IgG) is a major class of immunoglobulins found in the blood, including many of the most common antibodies circulating in the blood. Immunoglobulin A (IgA) is an antibody present in small amounts in blood and is the predominant antibody class found in the mucosal surfaces of the lung and the gastrointestinal tracts. Table 5 and Table 6 identify numerous target antigens associated with IgG and IgA, respectively, that are bound more often by antibodies from sera from lung adenocarcinoma individuals than by antibodies from healthy individuals.

One embodiment of the invention is a method of detecting autoantibodies in a test sample from an individual suspected of having lung adenocarcinoma by contacting the test sample from the individual with one or more target antigens each comprising an autoantigen of Table 1, Table 3, Table 4, Table 5 or Table 6 (provided below) or a fragment thereof comprising an epitope; and detecting binding of the one or more target antigens, wherein the binding of the one or more target antigens detects the presence of the one or more antibodies in the test sample. In a further embodiment, at least 10%; at least 25%; at least 50%; at least 80%; or at least 95% of the target antigens are bound by one or more antibodies from the test sample. The sample used in the detection and diagnosis methods of the invention can be any type of sample, but preferably is a saliva sample or a blood sample, or a fraction thereof, such as plasma or serum. Optionally, the test sample is taken from the individual prior to pathology confirmed diagnosis of lung adenocarcinoma. In a further embodiment, the one or more target antigens comprise one or more antigens selected from the group consisting of COQ3,LSM8, STAU, WDR27-A, WTAP-A, HEXIM1-A, and AHNAK-A.

Another embodiment of the present invention is a composition comprising one or more human antibodies from an individual with lung adenocarcinoma, wherein each antibody is bound to one or more target antigens each comprising an autoantigen of Table 1 or fragments thereof comprising an epitope. The target antigens may be immobilized on a solid support or may be part of a protein microarray. Another embodiment of the present invention is a solid support comprising two or more target antigens each comprising an autoantigen of Table 1 or fragments thereof comprising an epitope; and an immobilized human antibody control, wherein the human antibody control is a positive control for immunodetection.

The invention also provides kits that include one or more test antigens or one or more target antigens provided herein. The kits can include one or more reagents for detecting binding of an antibody from a sample. In some embodiments, the one or more test antigens or one or more target antigens of a kit are provided bound to a solid support. The invention includes kits that include biomarker detection panels of the invention, including biomarker detection panels in which the target antigens are bound to one or more solid supports. In some embodiments of kits, the kit provides a biomarker detection panel in which the target antigens of the detection panel are bound to a chip or array.

In some embodiments, the invention provides compositions, kits and methods for detecting one or more identified biomarkers as a diagnostic indicator for lung cancer, such as lung adenocarcinoma. Collection of blood samples or other test samples from a patient is routine and inexpensive. Additional uses of the invention include, among others: 1) the detection of one or more identified antigen biomarkers as a tool to select an appropriate therapeutic approach for treatment of a patient with lung adenocarcinoma; 2) the use of one or more detected biomarkers as a vaccine candidate or therapeutic target; 3) the use of one or more identified biomarkers as a screening tool for use in the development of new therapeutics including antibodies; 4) the use of one or more identified biomarkers to monitor the efficacy of a treatment on lung adenocarcinoma; and 5) the use of one or more identified biomarkers to monitor the progression of lung adenocarcinoma.

### DETAILED DESCRIPTION OF THE INVENTION

The invention is based on the identification of autoantigens for lung adenocarcinoma. Serum samples from healthy individuals as well as patients with lung cancer were profiled on ProtoArray® human protein micoarrays (Invitrogen Corporation, Carlsbad, Calif.), to identify multiple disease-specific biomarkers. The extensive content of the arrays, including lower abundance proteins, native conformation, and insect cell-derived post-translational modifications, enabled the identification of biomarkers not previously known to be associated with lung adenocarcinoma.

A list of antigen biomarkers (profiled using ProtoArray® human protein micoarrays) that were bound by antibodies from sera from patients diagnosed with lung adenocarcinoma is shown in Table 1. Lists of antigen biomarkers associated with IgG and IgA are provided in Table 5 and Table 6, respectively. Microarrays, or other assay formats, containing one or more of these biomarkers are able to detect the presence of antibodies in a patient sample that bind the biomarkers, enabling the detection, diagnosis and monitoring of the specific disease.

One embodiment of the present invention is a method of detecting one or more target antibodies in a test sample of an individual suspected of having lung adenocarcinoma comprising: a) contacting the test sample from the individual with one or more target antigens each comprising an autoantigen of Table 1, Table 5 or Table 6 or a fragment thereof comprising an epitope; and b) detecting binding of the one or more target antigens, wherein the binding of the one or more target antigens detects the presence of the one or more target antibodies in the test sample. In a further embodiment, the test sample is contacted with two or more; three or more, four or more, five or more, six or more, seven or more, eight or more, nine or more, ten or more; twenty or more; fifty or more; or all of the autoantigens of Table 1 or fragments thereof comprising an epitope. In a further embodiment, the quantitative amount of antibodies that bind to each biomarker is determined.

In a further embodiment, at least 1, 2, 3, 4, 5, 10, 20, 35, 50, 65, 75 or 90 antigen biomarkers must be bound by an antibody from the test sample to indicate the presence of lung adenocarcinoma. In a further embodiment, a kit and a method for diagnosing lung adenocarcinoma comprises contacting a test sample with one or more autoantigens, wherein one of the biomarkers is COQ3,LSM8, STAU, WDR27-A, WTAP-A, HEXIM1-A, AHNAK-A or fragments thereof comprising an epitope.

Lung adenocarcinoma will cause one or more of the target antigens identified in this invention to be present in altered amounts in the cells, tissues or bodily fluids of an individual. This includes elevated levels or decreased levels of the antigen compared to a healthy individual. One embodiment of the invention is a method of detecting altered levels of one or more target antibodies in a test sample of an individual compared to a healthy individual. Table 3 is a list of target antigens that were upregulated in patients diagnosed with lung adenocarcinoma compared to normal individuals. Table 4 is a list of target antigens that were downregulated in patients diagnosed with lung adenocarcinoma compared to normal individuals. The levels of one or more target antigens can further be elevated or decreased as the disease progresses. Thus, an individual in a later stage of the disease may exhibit altered amounts of one or more target antigens compared to the same individual or another individual at an earlier stage of the disease.

The progression or remission of a disease can be monitored by contacting test samples from an individual taken at different times with the panel of antigens. For example, a second test sample is taken from the patient and contacted with the antigen panel days or weeks after a first test sample. Alternatively, the second or subsequent test samples can be taken from the patient and tested against the panel of antigens at regular intervals, such as daily, weekly, monthly, quarterly, semi-annually, or annually. By testing the patient's test samples at different times, the presence of antibodies and therefore the stage of the disease can be compared. A further embodiment of the invention is a method of monitoring one or more target antibodies in test samples from an individual diagnosed as having lung adenocarcinoma comprising: a) contacting a first test sample from the individual with a first set of one or more target antigens; b) detecting binding of the one or more target antigens, wherein the binding of the one or more target antigens detects the presence of the one or more target antibodies in the first test sample; c) contacting a second test sample from the individual with a second set of the one or more target antigens; d) detecting binding of the one or more target antigens, wherein the binding of the one or more target antigens detects the presence of the one or more target antibodies in the second test sample; and e) comparing the presence of the one or more antibodies bound against the one or more target antigens from the first test sample with the one or more antibodies bound against the one or more target antigens from the second test sample, wherein each of the one or more target antigens comprises an autoantigen of Table 1 or fragments thereof comprising an epitope.

The progression of the disease is further monitored by quantitatively comparing the amounts of antibodies that bind to the autoantigens. Accordingly, another embodiment of the invention further comprises detecting the amount of the one or more antibodies against the one or more antigens in the first test sample and the second test sample; and comparing the amount of the one or more antibodies from the first test sample with the amount of the one or more antibodies from the second test sample.

Another embodiment of the invention is a mixture comprising one or more target antigens each comprising an autoantigen of Table 1 or a fragment thereof comprising an epitope; and a test sample from an individual suspected of having lung adenocarcinoma. The mixture optionally further comprises a control antibody against one or more of the target antigens. In a further embodiment, the mixture comprises two or more; ten or more; twenty or more; fifty or more; seventy-five or more; or all of the autoantigens of Table 1 or fragments thereof comprising an epitope. The test sample includes, but is not limited to, cells, tissues, or bodily fluids from an individual.

Another embodiment of the invention comprises a method of monitoring one or more target antibodies in test samples from an individual receiving treatment for lung adenocarcinoma comprising a) contacting a first test sample from an individual with a first set of one or more target antigens; b) detecting binding of the one or more target antigens to one or more antibodies in the first test sample, wherein the presence of the one or more antibodies bound against the one or more target antigens detects the one or more target antibodies; c) administering a treatment for lung adenocarcinoma to the individual; d) after the administration of the treatment, contacting a second test sample from the individual with a second set of the one or more target antigens; e) detecting binding of the one or more target antigens to one or more antibodies in the second test sample, wherein the presence of the one or more antibodies bound against the one or more target antigens detects the one or more target antibodies; and f) comparing the presence of the one or more antibodies against the one or more target antigens from the first sample with the one or more antibodies against the one or more target antigens from the second sample, wherein each of the one or more target antigens comprises an autoantigen of Table 1 or fragments thereof comprising an epitope.

By administering treatment, it is meant to encompass any therapeutic drug, procedure, or combination thereof administered to a patient to alleviate or treat lung adenocarcinoma, including, but not limited to, administering a drug orally or intravenously to a patient. The treatment may be continuous, that is, administered to the patient at regular intervals. Multiple test samples can be taken from the patient during the course of the treatment. Preferably, the first test sample is taken from the patient before treatment begins.

In a further embodiment, the amount of the one or more antibodies against the one or more antigens in each test sample is detected; and the amount of the one or more antibodies from the first test sample is compared with the amount of one or more antibodies from the second test sample.

The invention also provides a method of staging lung adenocarcinoma in an individual. This method comprises identifying a human patient having lung adenocarcinoma and analyzing cells, tissues or bodily fluid from such human patient for the biomarkers of the present invention associated with lung adenocarcinoma. The presence or level of the biomarker is then compared to the level of the biomarker in the same cells, tissues or bodily fluid type of a healthy control individual, or with a reference range of the level of biomarker obtained from at least one healthy control individual. An elevated level of immune reactivity against a biomarker protein identified as being present in elevated amounts in lung adenocarcinoma patients, when compared to the control or reference range, is associated with the presence of lung adenocarcinoma in the test individual. A decreased level of immune reactivity against a biomarker protein identified as being present in decreased amounts in lung adenocarcinoma patients, when compared to the control or reference range, is associated with the presence of lung adenocarcinoma in the test individual.

### DEFINITIONS

The term “about” as used herein refers to a value within 10% of the underlying parameter (i.e., plus or minus 10%), and is sometimes a value within 5% of the underlying parameter (i.e., plus or minus 5%), a value sometimes within 2.5% of the underlying parameter (i.e., plus or minus 2.5%), or a value sometimes within 1% of the underlying parameter (i.e., plus or minus 1%), and sometimes refers to the parameter with no variation. Thus, a distance of “about 20 nucleotides in length” includes a distance of 19 or 21 nucleotides in length (i.e., within a 5% variation) or a distance of 20 nucleotides in length (i.e., no variation) in some embodiments.

As used herein, the article “a” or “an” can refer to one or more of the elements it precedes (e.g., a protein microarray “a” protein may comprise one protein sequence or multiple proteins).

The term “or” is not meant to be exclusive to one or the terms it designates. For example, as it is used in a phrase of the structure “A or B” may denote A alone, B alone, or both A and B.

By “biomarker” it is meant a biochemical characteristic that can be used to detect, diagnose, prognose, direct treatment, or to measure the progress of a disease or condition, or the effects of treatment of a disease or condition. Biomarkers include, but are not limited to, the presence of a nucleic acid, protein, carbohydrate, antibody, or combinations thereof, associated with the presence of a disease in an individual. The present invention provides biomarkers for cancer, specifically lung adenocarcinoma, that are antibodies present in the sera of subjects diagnosed with lung adenocarcinoma. The biomarker antibodies in the present invention are the autoantibodies displaying increased reactivity in individuals with lung adenocarcinoma, most likely as a consequence of their increased abundance. The autoantibodies can be detected with autoantigens, human proteins that are specifically bound by the antibodies. Importantly, biomarkers need not be expressed in a majority of disease individuals to have clinical value. The receptor tyrosine kinase Her2 is known to be over-expressed in just approximately 25% of all breast cancers (J. S. Ross et al., Mol Cell Proteomics 3, 379-98 (April, 2004)), and yet is a clinically important indicator of disease progression as well as specific therapeutic options.

“Biomolecule” refers to an organic molecule of biological origin, e.g., steroids, fatty acids, amino acids, nucleotides, sugars, peptides, polypeptides, antibodies, polynucleotides, complex carbohydrates or lipids.

The phrase “differentially present” refers to differences in the quantity of a biomolecule (such as an antibody) present in a sample taken from patients having lung adenocarcinoma as compared to a comparable sample taken from patients who do not have cancer (e.g., normal or healthy patients). A biomolecule is differentially present between the two samples if the amount of the polypeptide in one sample is significantly different from the amount of the polypeptide in the other sample. For example, a polypeptide is differentially present between the two samples if it is present in an amount (e.g., concentration, mass, molar amount, etc.) at least about 150%, at least about 200%, at least about 500% or at least about 1000% greater or lesser than it is present in the other sample, or if it is detectable (gives a signal significantly greater than background or a negative control) in one sample and not detectable in the other. Any biomolecules that are differentially present in samples taken from lung adenocarcinoma patients as compared to subjects who do not have lung adenocarcinoma can be used as biomarkers.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′.sub.2 fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region. An “autoantibody” is an antibody that is directed against the host's own proteins or other molecules. In the present invention, high throughput microarrays have been used to detect autoantibodies from lung adenocarcinoma patients that are not typically present in normal patients.

The term “antigen” or “test antigen” as used herein refers to proteins or polypeptides to be used as targets for screening test samples obtained from subjects for the presence of autoantibodies. “Autoantigen” is used to denote antigens for which the presence of antibodies in a sample of an individual has been detected. These antigens, test antigens, or autoantigens are contemplated to include any fragments thereof of the so-identified proteins, in particular, immunologically detectable fragments. They are also meant to include immunologically detectable products of proteolysis of the proteins, as well as processed forms, post-translationally modified forms, such as, for example, “pre,” “pro,” or “prepro” forms of markers, or the “pre,” “pro,” or “prepro” fragment removed to form the mature marker, as well as allelic variants and splice variants of the antigens, test antigens, or autoantigens. The identification or listing of antigens, test antigens, and autoantigens also includes amino acid sequence variants of these, for example, sequence variants that include a fragment, domain, or epitope that shares immune reactivity with the identified antigen, test antigen, and autoantigen protein. Similarly, an “autoantigen” refers to a molecule, such as a protein, endogenous to the host that is recognized by an autoantibody.

An “epitope” is a site on an antigen, such as an autoantigen disclosed herein, recognized by an antibody.

As used herein, the word “protein” refers to a full-length protein, a portion of a protein, or a peptide. Proteins can be produced via fragmentation of larger proteins, or chemically synthesized. Proteins may, for example, be prepared by recombinant overexpression in a species such as, but not limited to, bacteria, yeast, insect cells, and mammalian cells. Proteins to be placed in a protein microarray of the invention, may be, for example, are fusion proteins, for example with at least one affinity tag to aid in purification and/or immobilization. In certain aspects of the invention, at least 2 tags are present on the protein, one of which can be used to aid in purification and the other can be used to aid in immobilization. In certain illustrative aspects, the tag is a His tag, a GST tag, or a biotin tag. Where the tag is a biotin tag, the tag can be associated with a protein in vitro or in vivo using commercially available reagents (Invitrogen, Carlsbad, Calif.). In aspects where the tag is associated with the protein in vitro, a Bioease tag can be used (Invitrogen, Carlsbad, Calif.).

As used herein, the term “peptide,” “oligopeptide,” and “polypeptide” are used interchangeably with protein herein and refer to a sequence of contiguous amino acids linked by peptide bonds. As used herein, the term “protein” refers to a polypeptide that can also include post-translational modifications that include the modification of amino acids of the protein and may include the addition of chemical groups or biomolecules that are not amino acid-based. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

A “variant” of a polypeptide or protein, as used herein, refers to an amino acid sequence that is altered with respect to the referenced polypeptide or protein by one or more amino acids. In the present invention, a variant of a polypeptide retains the antigenicity, or antibody-binding property, of the referenced protein. In preferred aspects of the invention, a variant of a polypeptide or protein can be bound by the same population of autoantibodies that are able to bind the referenced protein. Preferably a variant of a polypeptide has at least 60% identity to the referenced protein over a sequence of at least 15 amino acids. More preferably a variant of a polypeptide is at least 70% identical to the referenced protein over a sequence of at least 15 amino acids. Protein variants can be, for example, at least 80%, at least 90%, at least 95%, or at least 99% identical to referenced polypeptide over a sequence of at least 15 amino acids. Protein variants of the invention can be, for example, at least 80%, at least 90%, at least 95%, or at least 99% identical to referenced polypeptide over a sequence of at least 20 amino acids. The variant may have “conservative” changes, wherein a substituted amino acid has similar structural or chemical properties (e.g., replacement of leucine with isoleucine). A variant may also have “nonconservative” changes (e.g., replacement of glycine with tryptophan). Analogous minor variations may also include amino acid deletions or insertions, or both. Guidance in determining which amino acid residues may be substituted, inserted, or deleted without abolishing immunological reactivity may be found using computer programs well known in the art, for example, DNASTAR software.

Protein biomarkers used in a protein array of the present invention may be the full protein or fragments of the full protein. Protein fragments are suitable for use as part of the protein array as long as the fragments contain the epitope recognized by the antibodies. The required epitope for a given full protein can be mapped using protein microarrays, and with ELISPOT or ELISA techniques. It is understood that the antigen biomarkers provided by the present invention are meant to encompass the full protein as well as fragments thereof comprising an epitope. Typically, suitable protein fragments comprise at least 5%; at least 10%; at least 20%; or at least 50% of the full length protein amino acid sequence. In one embodiment of the present invention, protein fragments of target autoantigens contain at least 5 contiguous amino acids, at least 6 contiguous amino acids; at least 7 contiguous amino acids, at least 8 contiguous amino acids, at least 9 contiguous amino acids, at least 10 contiguous amino acids; at least 20 contiguous amino acids; at least 25 contiguous amino acids, at least 50 contiguous amino acids; at least 100 contiguous amino acids; or at least 200 contiguous amino acids of the full length protein.

As used herein, a “biomarker detection panel” or “biomarker panel” refers to a set of biomarkers that are provided together for detection, diagnosis, prognosis, staging, or monitoring of a disease or condition, based on detection values for the set (panel) of biomarkers.

The methods of the present invention are carried out on test samples derived from patients, including individuals suspected of having lung adenocarcinoma and those who have been diagnosed to have the disease. A “test sample” as used herein can be any type of sample, such as a sample of cells or tissue, or a sample of bodily fluid, preferably from an animal, most preferably a human. The sample can be a tissue sample, such as a swab or smear, or a pathology or biopsy sample of tissue, including tumor tissue. Samples can also be tissue extracts, for example from tissue biopsy or autopsy material. A sample can be a sample of bodily fluids, such as but not limited to blood, plasma, serum, sputum, semen, synovial fluid, cerebrospinal fluid, urine, lung aspirates, nipple aspirates, tears, or a lavage. Samples can also include, for example, cells or tissue extracts such as homogenates, cell lysates or solubilized tissue obtained from a patient. A preferred sample is a blood or serum sample.

By “blood” is meant to include whole blood, plasma, serum, or any derivative of blood. A blood sample may be, for example, serum.

A “patient” is an individual diagnosed with a disease or being tested for the presence of disease. A patient tested for a disease can have one or more indicators of a disease state, or can be screened for the presence of disease in the absence of any indicators of a disease state. As used herein an individual “suspected” of having a disease can have one or more indicators of a disease state or can be part of a population routinely screened for disease in the absence of any indicators of a disease state.

By “an individual suspected of having lung adenocarcinoma,” is meant an individual who has been diagnosed with lung adenocarcinoma, or who has at least one indicator of lung adenocarcinoma, or who is at an increased risk of developing lung adenocarcinoma due to age, gender, genetic factors, a history of smoking, environmental and/or nutritional factors.

As used herein, the term “array” refers to an arrangement of entities in a pattern on a substrate. Although the pattern is typically a two-dimensional pattern, the pattern may also be a three-dimensional pattern. In a protein array, the entities are proteins. In certain embodiments, the array can be a microarray or a nanoarray. A “nanoarray” is an array in which separate entities are separated by 0.1 nm to 10 μm, for example from 1 nm to 1 μm. A “microarray” is an array in the density of entities on the array is at least 100/cm². On microarrays separate entities can be separated, for example, by more than 1 μm.

The term “protein array” as used herein refers to a protein array, a protein microarray or a protein nanoarray. A protein array may include, for example, but is not limited to, a “ProtoArray®” protein high density array (Invitrogen, Carlsbad, Calif., available on the Internet at Invitrogen.com). The ProtoArray® high density protein array can be used to screen complex biological mixtures, such as serum, to assay for the presence of autoantibodies directed against human proteins. Alternatively, a custom protein array that includes autoantigens, such as those provided herein, for the detection of autoantibody biomarkers, can be used to assay for the presence of autoantibodies directed against human proteins. In certain disease states including autoimmune diseases and cancer, autoantibodies are expressed at altered levels relative to those observed in healthy individuals.

The term “protein chip” is used in the present application synonymously with protein array or microarray.

The phrase “diagnosis” as used herein refers to methods by which the skilled artisan can estimate and/or determine whether or not a patient is suffering from a given disease or condition. The skilled artisan often makes a diagnosis on the basis of one or more diagnostic indicators, i.e., a marker, the presence, absence, or amount of which is indicative of the presence, severity, or absence of the condition, physical features (lumps or hard areas in or on tissue), or histological or biochemical analysis of biopsied or sampled tissue or cells, or a combination of these.

Preferably, the test sample is taken from the individual and screened prior to pathology confirmed diagnosis of lung adenocarcinoma. The term “pathology confirmed diagnosis of lung adenocarcinoma” as used herein refers to a diagnosis of lung adenocarcinoma using routine physical features, such as presence of a tumor mass, lumps or hard areas in or on tissue, and physical symptoms such as coughing, bloody sputum, and difficulty breathing. Preferably, the individual is screened for lung adenocarcinoma before noticeable symptoms appear and before metastasis.

Similarly, a prognosis is often determined by examining one or more “prognostic indicators”, the presence or amount of which in a patient (or a sample obtained from the patient) signal a probability that a given course or outcome will occur. For example, when one or more prognostic indicators reach a sufficiently high level in samples obtained from such patients, the level may signal that the patient is at an increased probability of having a disease or condition in comparison to a similar patient exhibiting a lower marker level. A level or a change in level of a prognostic indicator, which in turn is associated with an increased probability of morbidity or death, is referred to as being “associated with an increased predisposition to an adverse outcome” in a patient. For example, preferred prognostic markers can predict the onset of lung adenocarcinoma in a patient with one or more target antibodies of Table 1, or a more advanced stage of lung adenocarcinoma in a patient diagnosed with the disease.

The term “correlating,” as used herein in reference to the use of diagnostic and prognostic indicators, refers to comparing the presence or amount of the indicator in a patient to its presence or amount in persons known to suffer from, or known to be at risk of, a given condition; or in persons known to be free of a given condition. As discussed above, a marker level in a patient sample can be compared to a level known to be associated with lung adenocarcinoma. The sample's marker level is said to have been correlated with a diagnosis; that is, the skilled artisan can use the marker level to determine whether the patient has lung adenocarcinoma, and respond accordingly. Alternatively, the sample's marker level can be compared to a marker level known to be associated with a good outcome (e.g., the absence of lung adenocarcinoma, etc.). In preferred embodiments, a profile of marker levels are correlated to a global probability or a particular outcome using ROC curves.

The phrase “determining the prognosis” as used herein refers to methods by which the skilled artisan can predict the course or outcome of a condition in a patient. The term “prognosis” does not refer to the ability to predict the course or outcome of a condition with 100% accuracy, or even that a given course or outcome is more likely to occur than not. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. For example, in individuals not exhibiting the condition, the chance of a given outcome may be about 3%. In preferred embodiments, a prognosis is about a 5% chance of a given outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−1%.

“Diagnostic” means identifying the presence or nature of a pathologic condition. Diagnostic methods differ in their sensitivity and specificity. While a particular diagnostic method may not provide a definitive diagnosis of a condition, it suffices if the method provides a positive indication that aids in diagnosis.

“Sensitivity” is defined as the percent of diseased individuals (individuals with lung adenocarcinoma) in which the biomarker of interest is detected (true positive number/total number of diseased×100). Nondiseased individuals diagnosed by the test as diseased are “false positives”.

“Specificity” is defined as the percent of nondiseased individuals for which the biomarker of interest is not detected (true negative/total number without disease x 100). Diseased individuals not detected by the assay are “false negatives.” Subjects who are not diseased and who test negative in the assay, are termed “true negatives.”

A “diagnostic amount” of a marker refers to an amount of a marker in a subject's sample that is consistent with a diagnosis of lung adenocarcinoma. A diagnostic amount can be either in absolute amount (e.g., X nanogram/ml) or a relative amount (e.g. relative intensity of signals).

A “test amount” of a marker refers to an amount of a marker present in a sample being tested. A test amount can be either in absolute amount (e.g., X nanogram/ml) or a relative amount (e.g., relative intensity of signals).

A “control amount” of a marker can be any amount or a range of amount which is to be compared against a test amount of a marker. For example, a control amount of a marker can be the amount of a marker (e.g., seminal basic protein) in an autoimmune disease patient, cancer patient or a normal patient. A control amount can be either in absolute amount (e.g., X nanogram/ml) or a relative amount (e.g., relative intensity of signals).

“Detect” refers to identifying the presence, absence or amount of the object to be detected.

“Label” or a “detectable moiety” refers to a composition detectable by spectroscopic, photochemical, biochemical, immunochemical, or chemical means. For example, useful labels include radiolabels such as ³²P, ³⁵S, or ¹²⁵I; fluorescent dyes; chromophores, electron-dense reagents; enzymes that generate a detectable signal (e.g., as commonly used in an ELISA); or spin labels. The label or detectable moiety has or generates a measurable signal, such as a radioactive, chromogenic, or fluorescent signal, that can be used to quantify the amount of bound detectable moiety in a sample. The detectable moiety can be incorporated in or attached to a primer or probe either covalently, or through ionic, van der Waals or hydrogen bonds, e.g., incorporation of radioactive nucleotides, or biotinylated nucleotides that are recognized by streptavidin. The label or detectable moiety may be directly or indirectly detectable. Indirect detection can involve the binding of a second directly or indirectly detectable moiety to the detectable moiety. For example, the detectable moiety can be the ligand of a binding partner, such as biotin, which is a binding partner for streptavidin, or a nucleotide sequence, which is the binding partner for a complementary sequence, to which it can specifically hybridize. The binding partner may itself be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules. (See, e.g., P. D. Fahrlander and A. Klausner, Bio/Technology 6:1165 (1988)). Quantitation of the signal is achieved by, e.g., scintillation counting, densitometry, or flow cytometry.

“Measure” in all of its grammatical forms, refers to detecting, quantifying or qualifying the amount (including molar amount), concentration or mass of a physical entity or chemical composition either in absolute terms in the case of quantifying, or in terms relative to a comparable physical entity or chemical composition.

“Immunoassay” is an assay in which an antibody specifically binds an antigen to provide for the detection and/or quantitation of the antibody or antigen. An immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Immune reactivity” as used herein means the presence or level of binding of an antibody or antibodies in a sample to one or more target antigens. A “pattern of immune reactivity” refers to the profile of binding of antibodies in a sample to a plurality of target antigens.

As used herein, “target antigen” refers to a protein, or to a portion, fragment, variant, isoform, processing product thereof having immunoreactivity of the protein, that is used to determine the presence, absence, or amount of an antibody in a sample from a subject. A “test antigen” is a protein evaluated for use as a target antigen. A test antigen is therefore a candidate target antigen, or a protein used to determine whether a portion of a test population has antibodies reactive against it. Use of the terms “target antigen”, “test antigen”, “autoantigen”, and, simply, “antigen” is meant to include the complete wild type mature protein, or can also denote a precursor, processed form (including, a proteolytically processed or otherwise cleaved form) unprocessed form, post-translationally modified, or chemically modified form of the protein indicated, in which the target antigen, test antigen, or antigen retains or possesses the specific binding characteristics of the referenced protein to one or more autoantibodies of a test sample. The protein can have, for example, one or more modifications not typically found in the protein produced by normal cells, including aberrant processing, cleavage or degradation, oxidation of amino acid residues, atypical glycosylation pattern, etc. The use of the terms “target antigen”, “test antigen”, “autoantigen”, or “antigen” also include splice isoforms or allelic variants of the referenced proteins, or can be sequence variants of the referenced protein, with the proviso that the “target antigen”, “test antigen”, “autoantigen”, or “antigen” retains or possesses the immunological reactivity of the referenced protein to one or more autoantibodies of a test sample. Use of the term “target antigen”, “test antigen”, “autoantigen”, or simply “antigen” specifically encompasses fragments of a referenced protein (“antigenic fragments”) that have the antibody binding specificity of the reference protein.

### Methods

The invention provides, in one aspect, a method of detecting one or more target antibodies in a test sample from an individual. The method includes: contacting the test sample from the individual with one or more target antigens of the invention, each comprising an autoantigen of Table 1, or a fragment thereof that includes an epitope recognized by a target antibody; and detecting binding of one or more antibodies in the sample to one or more target antigens, thereby detecting the presence of the one or more target antibodies in the sample. The target antigen can be any of the target antigens provided in Table 1, or a fragment thereof that includes an epitope. Furthermore, the target antigen can be a panel of target antigens that includes, for example, 2, 3, 4, 5, 6, 7, 8, 9, 10, 15, 20, 25, 50, or all target antigens of Table 1. The method can be carried out using virtually any immunoassay method. Non-limiting examples of immunoassay methods are provided below.

In certain aspects, the target antigen(s) are one or more of the antigens of Table 1 or Table 2 that have a p-value t_test (N,C,1,3) of equal or less than 0.05, 0.04, 0.03, 0.02, 0.01, 0.005, or 0.001. In certain aspects, the target antigen(s) are one or more of the antigens of Table 1 or Table 2 that have an M-stat p-value of equal or less than 0.05, 0.04, 0.03, 0.02, 0.01, 0.005, or 0.001. In certain aspects, the method is a method for screening for lung cancer wherein the target antigen(s) are one or more of the antigens of Table 2 that have a p-value t_test (N,C,1,3) of equal or less than 0.05, 0.04, 0.03, 0.02, 0.01, 0.005, or 0.001. In certain aspects, the method is a method for screening for lung cancer wherein the target antigen(s) are one or more of the antigens of Table 2 that have an M-stat p-value of equal or less than 0.05, 0.04, 0.03, 0.02, 0.01, 0.005, or 0.001.

The individual from whom the test sample is taken can be any individual, healthy or suspected of having cancer, and in some embodiments is an individual that is being screened for lung adenocarcinoma.

Binding is typically detected using an immunoassay, which can be in various formats as described in detail below. Detection of binding in certain illustrative embodiments makes use of one or more solid supports to which the test antigen is immobilized on a substrate to which the sample from an individual, typically a human subject, is applied. After incubation of the sample with the immobilized antigen, or optionally, concurrently with the incubation of the sample, an antibody that is reactive against human antibodies (for example, an anti-human IgG antibody that is from a species other than human, for example, goat, rabbit, pig, mouse, etc.) can be applied to the solid support with which the sample is incubated. The non-human antibody is directly or indirectly labeled. After removing nonspecifically bound antibody, signal from the label that is significantly above background level is indicative of binding of a human antibody from the sample to a test antigen on the solid support.

In the methods provided herein, the sample can be any sample of cells or tissue, or of bodily fluid. Since the autoantibodies being screened for circulate in the blood an/d are fairly stable in blood sample, in certain illustrative embodiments, the test sample is blood or a fraction thereof, such as, for example, serum. The sample can be unprocessed prior to contact with the test antigen, or can be a sample that has undergone one or more processing steps. For example, a blood sample can be processed to remove red blood cells and obtain serum.

The test sample can be contacted with a test antigen provided in solution phase, or the test antigen can be provided bound to a solid support. In preferred embodiments, the detection is performed by an immunoassay, as described in more detail below. Detection of binding of the target sample to a test antigen indicates the presence of an autoantibody that specifically binds the test antigen in the sample. Identifying an autoantibody present in a sample from an individual can be used to identify biomarkers of a disease or condition, or to diagnose a disease or condition.

The detection can be performed on any solid support, such as a bead, dish, plate, well, sheet, membrane, slide, chip, or array, such as a protein array, which can be a microarray, and can optionally be a high density microarray.

The detection method can provide a positive/negative binding result, or can give a value that can be a relative or absolute value for the level of the autoantibody biomarker in the sample. The result can provide a diagnosis, prognosis, or be used as an indicator for conducting further tests or evaluation that may or may not result in a diagnosis or prognosis.

The method includes detecting more than one autoantibody in a sample from an individual, in which one or more of the test antigens used to detect autoantibodies is a test antigen of Table 1.

A fragment that includes an epitope recognized by an antibody can be 5, 6, 7, 8, 9, 10, 11, 12, 13, 14, 15, 20, 25, 30, 35, 40, 45, 50, 75, 100, 150, 200, 250, 300, 350, 400, 450, 500, 750, 1000 or more amino acids in length. The fragment can also be between 5, 10, 15, 20, 25, 50, 75, 100, 150, 200, or 250 amino acids less than the entire length of an autoantigen. Typically, such epitopes are characterized in advance such that it is known that autoantibodies for a given autoantigen recognize the epitope. Methods for epitope mapping are well known in the art.

In some embodiments, the detection is performed on a protein array, which can be a microarray, and can optionally be a microarray that includes proteins at a concentration of at least 100/cm² or 1000/cm², or greater than 400/cm².

The detection method can provide a positive/negative binding result, or can give a value that can be a relative or absolute value for the level of the autoantibody biomarker in the sample.

The method can be repeated over time, for example, to monitor a pre-disease state, to monitor progression of a disease, or to monitor a treatment regime. The results of a diagnostic test that determines the immune reactivity of a patient sample to a test antigen can be compared with the results of the same diagnostic test done at an earlier time. Significant differences in immune reactivity over time can contribute to a diagnosis or prognosis of adenocarcinoma.

In some preferred embodiments, the biomarker detection panel has an ROC/AUC of 0.550 or greater, of 0.600 or greater, 0.650 or greater, 0.700 or greater, 0.750 or greater, 0.800 or greater, 0.850 or greater, or 0.900 or greater for distinguishing between a normal state and a disease state in a subject.

A target antigen present in a biomarker detection panel can be an entire mature form of a protein, such as a protein referred to as a target antigen (for example, a target antigen listed in Table 1), or can be a precursor, processed form, unprocessed form, isoforms, variant, a fragment thereof that includes an epitope, or allelic variant thereof, providing that the modified, processed, or variant for of the protein has the ability to bind autoantigens present in samples from individuals.

In some embodiments, a biomarker detection panel used to detect lung adenocarcinoma comprises one or more target antigens of Table 1. In some embodiments, a biomarker detection panel used to detect lung adenocarcinoma comprises two or more target antigens of Table 1. In some embodiments, a biomarker detection panel used to detect lung adenocarcinoma comprises three or more target antigens of Table 1. In some embodiments, a biomarker detection panel used to detect lung adenocarcinoma comprises four or more target antigens of Table 1. In some embodiments, the test sample is contacted with a biomarker detection panel comprising five or more target antigens of Table 1. In some embodiments, the biomarker detection panel used in the methods of the invention includes six, seven, eight, nine, ten, eleven or twelve target antigens of Table 1. In some embodiments, the biomarker detection panel used in the methods of the invention includes 12, 13, 14, 15, 16, 17, 18, 19, 20, or more target antigens of Table 1. In some embodiments, the test sample is contacted with a biomarker detection panel comprising 21, 22, 23, 24, 25, 26, 27, 28, 29, or 30 antigens of Table 1. A biomarker detection panel can comprise between 30 and 35 antigens of Table 1, between 35 and 40 antigens of Table 1, between 40 and 45 antigens of Table 1, between 45 and 50 antigens of Table 1, between 50 and 55 antigens of Table 1, between 55 and 60 antigens of Table 1, between 60 and 65 antigens of Table 1, between 65 and 70 antigens of Table 1, between 70 and 75 antigens of Table 1, between 75 and 80 antigens of Table 1, between 80 and 85 antigens of Table 1, between 85 and 90 antigens of Table 1, between 90 and 95 antigens of Table 1, between 95 and 100 antigens of Table 1, between 100 and 105 antigens of Table 1, or between 105 and 108 antigens of Table 1.

In certain aspects, the target antigen(s) are one or more of the antigens of Table 1 or Table 2 that have a p-value t_test (N,C,1,3) of equal or less than 0.05, 0.04, 0.03, 0.02, 0.01, 0.005, or 0.001. In certain aspects, the target antigen(s) are one or more of the antigens of Table 1 or Table 2 that have an M-stat p-value of equal or less than 0.05, 0.04, 0.03, 0.02, 0.01, 0.005, or 0.001,

### Immunoassays

Virtually any immunoassay technique known in the art can be used to detect antibodies that bind an antigen according to methods and kits of the present invention. Such immunoassay methods include, without limitation, radioimmunoassays, immunohistochemistry assays, competitive-binding assays, Western Blot analyses, ELISA assays, sandwich assays, two-dimensional gel electrophoresis (2D electrophoresis) and non-gel based approaches such as mass spectrometry or protein interaction profiling, all known to those of ordinary skill in the art. These methods may be carried out in an automated manner, as is known in the art. Such immunoassay methods may also be used to detect the binding of antibodies in a sample to a target antigen.

In one example of an ELISA method, the method includes incubating a sample with a target protein and incubating the reaction product formed with a binding partner, such as a secondary antibody, that binds to the reaction product by binding to an antibody from the sample that associated with the target protein to form the reaction product. In some cases these may comprise two separate steps, in others, the two steps may be simultaneous, or performed in the same incubation step. Examples of methods of detection of the binding of the target protein to an antibody, is the use of an anti-human IgG (or other) antibody or protein A. This detection antibody may be linked to, for example, a peroxidase, such as horseradish peroxidase.

Using protein arrays for immunoassays allows the simultaneous analysis of multiple proteins. For example, target antigens or antibodies that recognize biomarkers that may be present in a sample are immobilized on microarrays. Then, the biomarker antibodies or proteins, if present in the sample, are captured on the cognate spots on the array by incubation of the sample with the microarray under conditions favoring specific antigen-antibody interactions. The binding of protein or antibody in the sample can then be determined using secondary antibodies or other binding labels, proteins, or analytes. Comparison of proteins or antibodies found in two or more different samples can be performed using any means known in the art. For example, a first sample can be analyzed in one array and a second sample analyzed in a second array that is a replica of the first array.

The term “sandwich assay” refers to an immunoassay where the antigen is sandwiched between two binding reagents, which are typically antibodies. The first binding reagent/antibody is attached to a surface and the second binding reagent/antibody comprises a detectable moiety or label. Examples of detectable moieties include, for example and without limitation: fluorochromes, enzymes, epitopes for binding a second binding reagent (for example, when the second binding reagent/antibody is a mouse antibody, which is detected by a fluorescently-labeled anti-mouse antibody), for example an antigen or a member of a binding pair, such as biotin. The surface may be a planar surface, such as in the case of a typical grid-type array (for example, but without limitation, 96-well plates and planar microarrays), as described herein, or a non-planar surface, as with coated bead array technologies, where each “species” of bead is labeled with, for example, a fluorochrome (such as the Luminex technology described herein and in U.S. Pat. Nos. 6,599,331, 6,592,822 and 6,268,222), or quantum dot technology (for example, as described in U.S. Pat. No. 6,306,610).

A variety of different solid phase substrates can be used to detect a protein or antibody in a sample, or to quantitate or determine the concentration of a protein or antibody in a sample. The choice of substrate can be readily made by those of ordinary skill in the art, based on convenience, cost, skill, or other considerations. Useful substrates include without limitation: beads, bottles, surfaces, substrates, fibers, wires, framed structures, tubes, filaments, plates, sheets, and wells. These substrates can be made from: polystyrene, polypropylene, polycarbonate, glass, plastic, metal, alloy, cellulose, cellulose derivatives, nylon, coated surfaces, acrylamide or its derivatives and polymers thereof, agarose, or latex, or combinations thereof. This list is illustrative rather than exhaustive.

Other methods of protein detection and measurement described in the art can be used as well. For example, a single antibody can be coupled to beads or to a well in a microwell plate, and quantitated by immunoassay. In this assay format, a single protein can be detected in each assay. The assays can be repeated with antibodies to many analytes to arrive at essentially the same results as can be achieved using the methods of this invention. Bead assays can be multiplexed by employing a plurality of beads, each of which is uniquely labeled in some manner. For example each type of bead can contain a pre-selected amount of a fluorophore. Types of beads can be distinguished by determining the amount of fluorescence (and/or wavelength) emitted by a bead. Such fluorescently labeled beads are commercially available from Luminex Corporation (Austin, Tex.; see the worldwide web address of luminexcorp.com). The Luminex assay is very similar to a typical sandwich ELISA assay, but utilizes Luminex microspheres conjugated to antibodies or proteins (Vignali, J. Immunol. Methods 243:243-255 (2000)).

The methodology and steps of various antibody assays are known to those of ordinary skill in the art. Additional information may be found, for example, in Antibodies: A Laboratory Manual, Cold Spring Harbor Laboratory, Chap. 14 (1988); Bolton and Hunter, “Radioimmunoassay and Related Methods,” in Handbook of Experimental Immunology (D. M. Weir, ed.), Blackwell Scientific Publications, 1996; and Current Protocols in Immunology, (John E. Coligan, et al., eds) (1993).

The antibodies used to perform the foregoing assays can include polyclonal antibodies, monoclonal antibodies and fragments thereof as described supra. Monoclonal antibodies can be prepared according to established methods (see, e.g., Kohler and Milstein (1975) Nature 256:495; and Harlow and Lane (1988) Antibodies: A Laboratory Manual (C.H.S.P., N.Y.)).

An antibody can be a complete immunoglobulin or an antibody fragment. Antibody fragments used herein, typically are those that retain their ability to bind an antigen. Antibodies subtypes include IgG, IgM, IgA, IgE, or an isotype thereof (e.g., IgG1, IgG2a, IgG2b or IgG3). Antibody preparations can by polyclonal or monoclonal, and can be chimeric, humanized or bispecific versions of such antibodies. Antibody fragments include but are not limited to Fab, Fab′, F(ab)′2, Dab, Fv and single-chain Fv (ScFv) fragments. Bifunctional antibodies sometimes are constructed by engineering two different binding specificities into a single antibody chain and sometimes are constructed by joining two Fab′ regions together, where each Fab′ region is from a different antibody (e.g., U.S. Pat. No. 6,342,221). Antibody fragments often comprise engineered regions such as CDR-grafted or humanized fragments. Antibodies sometimes are derivitized with a functional molecule, such as a detectable label (e.g., dye, fluorophore, radioisotope, light scattering agent (e.g., silver, gold)) or binding agent (e.g., biotin, streptavidin), for example.

In certain embodiments, one or more diagnostic (or prognostic) biomarkers, such as one or more autoantibody biomarkers, are correlated to a condition or disease by the presence or absence of the biomarker(s). In other embodiments, threshold level(s) of a diagnostic or prognostic biomarker(s) can be established, and the level of the biomarker(s) in a sample can simply be compared to the threshold level(s).

As will be understood, for any particular biomarker, a distribution of biomarker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a biomarker changes with the disease) the test is considered to be abnormal and below which the test is considered to be normal. Receiver Operating Characteristic curves, or “ROC” curves, are typically generated by plotting the value of a variable versus its relative frequency in “normal” and “disease” populations. The area under the ROC curve is a measure of the probability that the perceived measurement will allow correct identification of a condition. ROC curves can also be generated using relative, or ranked, results. Methods of generating ROC curves and their use are well known in the art. See, e.g., Hanley et al., Radiology 143: 29-36 (1982).

One or more test antigens may have relatively low diagnostic or prognostic value when considered alone, but when used as part of a panel that includes other reagents for biomarker detection (such as but not limited to other test antigens), such test antigens can contribute to making a particular diagnosis or prognosis. In preferred embodiments, particular threshold values for one or more test antigens in a biomarker detection panel are not relied upon to determine if a profile of marker levels obtained from a subject are indicative of a particular diagnosis or prognosis. Rather, the present invention may utilize an evaluation of the entire marker profile of a biomarker detection panel, for example by plotting ROC curves for the sensitivity of a particular biomarker detection panel. In these methods, a profile of biomarker measurements from a sample of an individual is considered together to provide an overall probability (expressed either as a numeric score or as a percentage risk) that an individual has lung adenocarcinoma, for example. In such embodiments, an increase in a certain subset of biomarkers (such as a subset of biomarkers that includes one or more autoantibodies) may be sufficient to indicate a particular diagnosis (or prognosis) in one patient, while an increase in a different subset of biomarkers (such as a subset of biomarkers that includes one or more autoantibodies) may be sufficient to indicate the same or a different diagnosis (or prognosis) in another patient. Weighting factors may also be applied to one or more biomarkers being detected. As one example, when a biomarker is of particularly high utility in identifying a particular diagnosis or prognosis, it may be weighted so that at a given level it alone is sufficient to indicate a positive diagnosis. In another example, a weighting factor may provide that no given level of a particular marker is sufficient to signal a positive result, but only signals a result when another marker also contributes to the analysis.

In preferred embodiments, markers and/or marker panels are selected to exhibit at least 70% sensitivity, more preferably at least 80% sensitivity, even more preferably at least 85% sensitivity, still more preferably at least 90% sensitivity, and most preferably at least 95% sensitivity, combined with at least 70% specificity, more preferably at least 80% specificity, even more preferably at least 85% specificity, still more preferably at least 90% specificity, and most preferably at least 95% specificity. In particularly preferred embodiments, both the sensitivity and specificity are at least 75%, more preferably at least 80%, even more preferably at least 85%, still more preferably at least 90%, and most preferably at least 95%.

Using various subsets of the test antigens provided in Table 1, the present invention provides test antigens for detecting lung adenocarcinoma in a sample from an individual, and biomarker detection panels comprising combinations of the test antigens of Table 1 that can be used to detect and/or diagnose adenocarcinoma, specifically adenocarcinoma of the lung, with high sensitivity and specificity. Accordingly, methods, compositions, and kits are provided herein for the detection, diagnosis, staging, and monitoring of adenocarcinoma in individuals.

Automated systems for performing immunoassays, such as those utilized in the methods herein, are widely known and used in medical diagnostics. For example, random-mode or batch analyzer immunoassay systems can be used, as are known in the art. These can utilize magnetic particles or non-magnetic particles or microparticles and can utilize a fluorescence or chemiluminescence readout, for example. As non-limiting examples, the automated system can be an automated microarray hybridization station, an automated liquid handling robot, the Beckman ACCESS paramagnetic-particle, an chemiluminescent immunoassay, the Bayer ACS:180 chemiluminescent immunoassay or the Abbott AxSYM microparticle enzyme immunoassay. Such automated systems can be designed to perform methods provided herein for an individual antigen or for multiple antigens without multiple user interventions.

### Biomarker Detection Panels

The invention also provides biomarker detection panels for diagnosing, prognosing, monitoring, or staging lung adenocarcinoma, in which the biomarker detection panels comprise two or more target antigens selected from Table 1, in which at least 50% of the proteins of the test panel are proteins of Table 1. In some preferred embodiments, the proteins of the biomarker detection panel are provided on one or more solid supports, in which at least 50% of the proteins on the one or more solid supports to which the proteins of the panel are bound are of Table 1. Proteins of a biomarker detection panel can be provided bound to a solid support in the form of a bead, matrix, dish, well, plate, slide, sheet, membrane, filter, fiber, chip, or array. In some preferred embodiments, the proteins of the biomarker detection panel are provided on a protein array in which 50% or more of the proteins on the array are target antigens of the biomarker detection panel.

In certain aspects, the target antigen(s) are one or more of the antigens of Table 1 or Table 2 that have a p-value t_test (N,C,1,3) of equal or less than 0.05, 0.04, 0.03, 0.02, 0.01, 0.005, or 0.001. In certain aspects, the target antigen(s) are one or more of the antigens of Table 1 or Table 2 that have an M-stat p-value of equal or less than 0.05, 0.04, 0.03, 0.02, 0.01, 0.005, or 0.001,

The set of biomarkers in a biomarker detection panel are associated, either electronically, or preferably physically. For example, each biomarker of a biomarker detection panel can be provided in isolated form, in separate tubes that are sold and/or shipped together, for example as part of a kit. In certain embodiments, isolated biomarkers are formed into a detection panel by attaching them to the same solid support. The biomarkers of a biomarker panel can also be mixed together in the same solution.

The invention also provides biomarker detection panels for diagnosing, prognosing, monitoring, or staging lung adenocarcinoma, in which the biomarker detection panels comprise 2, 3, 4, 5, 6, 7, 8, 9, 10, 15, 20, 25, 30, 35, 40, 45, 50 or more target antigens selected from Table 1. In some preferred embodiments, the proteins of the biomarker detection panel are provided on one or more solid supports, in which at least 55%, 60%, 65%, 70%, 75%, 80%, 85%, 90%, 95% or 100% of the proteins on the one or more solid supports to which the proteins of the panel are bound are of Table 1. In some preferred embodiments, the proteins of the biomarker detection panel are provided on a protein array in which at least 55%, 60%, 65%, 70%, or 75%, 80%, 85%, 90%, 95% or 100% of the proteins on the array are target antigens of the biomarker detection panel.

In some embodiments, the biomarker detection panel used in the methods of the invention includes 6, 7, 8, 9, 10, 11, or 12 target antigens of Table 1. In some embodiments, the biomarker detection panel used in the methods of the invention includes 13, 14, 15, 16, 17, 18, 19, 20, or more target antigens of Table 1. In some embodiments, the test sample is contacted with a biomarker detection panel comprising 21, 22, 23, 24, 25, 26, 27, 28, 29, or 30 antigens of Table 1. A biomarker detection panel can comprise between 30 and 35 antigens of Table 1, between 35 and 40 antigens of Table 1, between 40 and 45 antigens of Table 1, between 45 and 50 antigens of Table 1, between 50 and 55 antigens of Table 1, between 55 and 60 antigens of Table 1, between 60 and 65 antigens of Table 1, between 65 and 70 antigens of Table 1, between 70 and 75 antigens of Table 1, between 75 and 80 antigens of Table 1, between 80 and 85 antigens of Table 1, between 85 and 90 antigens of Table 1, between 90 and 95 antigens of Table 1, between 95 and 100 antigens of Table 1, between 100 and 105 antigens of Table 1, or between 105 and 108 antigens of Table 1.

Also included in the invention is a composition that comprises a biomarker detection panel for diagnosing, prognosing, monitoring, or staging lung adenocarcinoma that comprises two or more target antigens selected from Table 1, in which at least one of the two or more target antigens is bound to an autoantibody from a sample of an individual. The invention also includes a biomarker detection panel for diagnosing, prognosing, monitoring, or staging lung adenocarcinoma that comprises 3, 4, 5, 6, 7, 8, 9, 10, 11, 12, 13, 14, 15, 16, 17, 18, 19, 20, 21, 22, 23, 24, 25, 26, 27, 28, 29, 30 or more target antigens selected from Table 1, in which at least one of the two or more target antigens is bound to an autoantibody from a sample of an individual. The arrays having bound antibody from a sample can be arrays in which at least 55%, 60%, 65%, 70%, 75%, 80%, 85%, 90%, of 95% of the protein bound to the arrays are proteins of Table 1.

### Method for Synthesizing Protein Antigens

The methods, kits, and systems provided herein include autoantigens, which typically are protein antigens. To obtain protein antigens to be used in the methods provided herein, known methods can be used for making and isolating viral, prokaryotic or eukaryotic proteins in a readily scalable format, amenable to high-throughput analysis. For example, methods include synthesizing and purifying proteins in an array format compatible with automation technologies. Therefore, in one embodiment, protein microarrays for the invention a method for making and isolating eukaryotic proteins comprising the steps of growing a eukaryotic cell transformed with a vector having a heterologous sequence operatively linked to a regulatory sequence, contacting the regulatory sequence with an inducer that enhances expression of a protein encoded by the heterologous sequence, lysing the cell, contacting the protein with a binding agent such that a complex between the protein and binding agent is formed, isolating the complex from cellular debris, and isolating the protein from the complex, wherein each step is conducted in a 96-well format.

In a particular embodiment, eukaryotic proteins are made and purified in a 96-array format (i.e., each site on the solid support where processing occurs is one of 96 sites), e.g., in a 96-well microtiter plate. In another embodiment, the solid support does not bind proteins (e.g., a non-protein-binding microtiter plate).

In certain embodiments, proteins are synthesized by in vitro translation according to methods commonly known in the art. For example, proteins can be expressed using a wheat germ, rabbit reticulocyte, or bacterial extract, such as the Expressway.

Any expression construct having an inducible promoter to drive protein synthesis can be used in accordance with the methods of the invention. The expression construct may be, for example, tailored to the cell type to be used for transformation. Compatibility between expression constructs and host cells are known in the art, and use of variants thereof are also encompassed by the invention.

In a particular embodiment, the fusion proteins have GST tags and are affinity purified by contacting the proteins with glutathione beads. In further embodiment, the glutathione beads, with fusion proteins attached, can be washed in a 96-well box without using a filter plate to ease handling of the samples and prevent cross contamination of the samples.

In addition, fusion proteins can be eluted from the binding compound (e.g., glutathione bead) with elution buffer to provide a desired protein concentration. In a specific embodiment, fusion proteins are eluted from the glutathione beads with 30 μl of elution buffer to provide a desired protein concentration.

For purified proteins that will eventually be spotted onto microscope slides, the glutathione beads are separated from the purified proteins. In one example, all of the glutathione beads are removed to avoid blocking of the microarrays pins used to spot the purified proteins onto a solid support. In one embodiment, the glutathione beads are separated from the purified proteins using a filter plate, for example, comprising a non-protein-binding solid support. Filtration of the eluate containing the purified proteins should result in greater than 90% recovery of the proteins.

The elution buffer may, for example, comprise a liquid of high viscosity such as, for example, 15% to 50% glycerol, for example, about 25% glycerol. The glycerol solution stabilizes the proteins in solution, and prevents dehydration of the protein solution during the printing step using a microarrayer.

Purified proteins may, for example, be stored in a medium that stabilizes the proteins and prevents desiccation of the sample. For example, purified proteins can be stored in a liquid of high viscosity such as, for example, 15% to 50% glycerol, for example, in about 25% glycerol. In one example, samples may be aliquoted containing the purified proteins, so as to avoid loss of protein activity caused by freeze/thaw cycles.

The skilled artisan can appreciate that the purification protocol can be adjusted to control the level of protein purity desired. In some instances, isolation of molecules that associate with the protein of interest is desired. For example, dimers, trimers, or higher order homotypic or heterotypic complexes comprising an overproduced protein of interest can be isolated using the purification methods provided herein, or modifications thereof. Furthermore, associated molecules can be individually isolated and identified using methods known in the art (e.g., mass spectroscopy).

The protein antigens once produced can be used in the biomarker panels, methods and kits provided herein as part of a “positionally addressable” array. The array includes a plurality of target antigens, with each target antigen being at a different position on a solid support. The array can include, for example 1, 2, 3, 4, 5, 8, 9, 10, 11, 12, 13, 14, 15, 16, 17, 18, 19, 20, 21, 22, 23, 24, 25, 26, 27, 28, 29, 30, 40, 50, 100, 200, 300, 400, or 500 different proteins. The array can include 1, 2, 3, 4, 5, 10, 15, 20, 25, 50, 75 or all the proteins of Table 1. In one aspect, the majority of proteins on an array include proteins identified as autoantigens that can have diagnostic value for a particular disease or medical condition when provided together autoantigen biomarker detection panel.

In one aspect, the protein array is a bead-based array. In another aspect, the protein array is a planar array. Methods for making protein arrays, such as by contact printing, are well known. In some embodiments, the detection is performed on a protein array, which can be a microarray, and can optionally be a microarray that includes proteins at a concentration of at least 100/cm² or 1000/cm², or greater than 400/cm².

### Kits

In certain embodiments of the invention, kits are provided. Thus, in some embodiments, a kit is provided that comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30-34, 35-39, 40-44, 45-49, 50-54, 55-59, 60-64, 65-69, 70-74, 75-79, 80-84, 85-89, 90-95 or more of the test antigen proteins provided in Table 1, Table 3, Table 4, Table 5 or Table 6 or a fragment thereof comprising an epitope recognized by a target antibody. In certain aspects the kit includes up to 10, 50, or 75 of the test antigen proteins of Table 1. A kit of the invention can include any of the biomarker detection panels disclosed herein.

In one embodiment, a kit for diagnosing lung adenocarcinoma comprises one or more, two or more, ten or more, twenty or more, fifty or more, or all of the autoantigens of Table 1 or a fragment thereof comprising an epitope; and means for detecting if one or more molecules in a test sample binds to one or more of the antigens. In some embodiments, the kits and protein arrays of the present invention contain less than 1,000 polypeptides, or less than 100 polypeptides.

In a further embodiment, the kit further comprises a control antibody against one or more of the antigens. The kit can include one or more positive controls, one or more negative controls, and/or one or more normalization controls.

The proteins of the kit may, for example, be immobilized on a solid support or surface. The proteins may, for example, be immobilized in an array. The protein microarray may use bead technology, such as the Luminex technology (Luminex Corp., Austin, Tex.). The test protein array may or may not be a high-density protein microarray that includes at least 100 proteins/cm². The kit can provide a biomarker detection panel of proteins as described herein immobilized on an array. At least 50%, at least 55%, at least 60%, at least 65%, at least 70%, at least 75%, at least 80%, at least 85%, at least 90%, or at least 95% of the proteins immobilized on the array can be proteins of the biomarker test panel. The array can include immobilized on the array one or more positive control proteins, one or more negative controls, and/or one or more normalization controls.

A kit may further comprise a reporter reagent to detect binding of human antibody to the proteins, such as, for example, an antibody that binds to human antibody, linked to a detectable label. A kit may further comprise reagents useful for various immune reactivity assays, such as ELISA, or other immunoassay techniques known to those of skill in the art. The assays in which the kit reagents can be used may be competitive assays, sandwich assays, and the label may be selected from the group of well-known labels used for radioimmunoassay, fluorescent or chemiluminescence immunoassay.

A kit can include reagents described herein in any combination. For example, in one aspect, the kit includes a biomarker detection panel as provided herein immobilized on a solid support and anti-human antibodies for detection in solution. The detection antibodies can comprise labels.

The kit can also include a program in computer readable form to analyze results of methods performed using the kits to practice the methods provided herein.

The kits of the present invention may also comprise one or more of the components in any number of separate containers, packets, tubes, vials, microtiter plates and the like, or the components may be combined in various combinations in such containers.

The kits of the present invention may also comprise instructions for performing one or more methods described herein and/or a description of one or more compositions or reagents described herein. Instructions and/or descriptions may be in printed form and may be included in a kit insert. A kit also may include a written description of an Internet location that provides such instructions or descriptions.

### EXAMPLES

The examples set forth below illustrate, but do not limit the invention.

### Example 1

In three separate studies, serum from twenty-three, twenty-one and nineteen normal control individuals and twenty-three, twenty-two and nineteen individuals with pathology confirmed lung adenocarcinoma were profiled against a high throughput human protein array. In a separate study, serum from thirty high risk patients one year prior to being diagnosed with lung adenocarcinoma and thirty normal control patients were also profiled against a high throughput human protein array. The serum samples were diluted 1:150 and used to probe human ProtoArray®. Specifically, arrays were blocked for 1 hour, incubated with dilute serum solution for 90 minutes, washed 3×10 minutes, incubated with anti-human IgG antibody conjugated to AlexaFluor 647 for 90 minutes, washed as above, dried, and scanned. Most of the serum samples were also tested using anti-human IgA antibody.

Following scanning, data was acquired using specialized software. Background-subtracted signals from each population were normalized utilizing a quantile normalization strategy and a lot-median-normalization strategy. All possible pairwise comparisons were performed between all groups of samples included in the study utilizing an M-statistics algorithm in which the M-statistic is identified that is associated with the lowest possible p-value for a particular pairwise comparison of sample populations.

Proteins of interest identified as significant interactors with antibodies present in the serum from lung adenocarcinoma patients are listed in Table 1 and again in Table 2.

### Example 2

The process for identifying biomarkers used in the invention was as follows.

First, the relative fluorescence unit values (RFUs) of the duplicate spots on the ProtoArray were averaged. Then, the RFUs for each antigen protein or protein fragment were normalized by lot-median-normalization (LMN). In LMN, the median signal within a lot was normalized to the average value among the lots used. Specifically, the median RFU value for each antigen protein within each lot of arrays was calculated. The medians for an antigen protein from several lots were averaged. Then, the RFUs of the antigen proteins on all the arrays within each lot were multiplied by the scalar: average antigen protein median/Lot-specific antigen protein median.

Next, the RFU values were quantile normalized. In quantile normalization, the median value of the highest signal on each of the arrays, the median value of the second highest signal on each of the arrays, the median value of the third highest signal on all of the arrays, etc. were determined. Then, the highest value on each array was assigned the value of the median of the highest values; the second highest signal on each array was assigned the value of the median of the second highest values, and so on.

Finally, antigen proteins that had exhibited a non-print event in one of the lots, or that exhibited vastly different reactivity between lots, were removed from consideration. This was done for each antigen protein by dividing the maximum median value among the lots used by the minimum median value among the lots used. If the ratio was greater than 5 or less then 0.2, the antigen protein was rejected for further consideration.

### Example 3

Serum samples from healthy individuals as well as individuals with lung adenocarcinoma were profiled on ProtoArray® human protein microarrays as described in Example 1. A number of potential antigen biomarkers were identified for lung adenocarcinoma. These proteins have the potential to serve as important diagnostic or prognostic indicators. Instead of an assay containing thousands or tens of thousands of proteins, a test sample can be profiled against an assay containing just the antigens associated with carcinoma, particularly lung adenocarcinoma.

Table 1 is a list of autoantigens that were bound more often by antibodies from sera from lung adenocarcinoma individuals than by antibodies from healthy individuals. Table 1 identifies antigens according to Genbank ID number for the nucleotide sequence that encodes the antigens. It is understood that an antigen of Table 1 refers to a protein or fragments thereof that is encoded by the nucleotide sequence associated with the nucleotide ID number.

TABLE 1
Patients diagnosed with lung adenocarcinoma vs. healthy patients

| | | | | | | | | BEST | |
|---|---|---|---|---|---|---|---|---|---|
| | | p- | | | | p- | BEST | M- | |
| | | value | BEST | BEST | | value | M- | Stat | |
| | | t_Test | M-Stat | M-Stat | BEST | t_Test | Stat | p- | BEST |
| UI1 | SYMBOL | N, C, 1, 3 | Prev | p-value | M-Stat | N, C, 1, 3 | Prev | value | M-Stat |
| BC000082A | ARS2-A | 0.028 | 16.1% | 0.001 | M3 = 14 | 0.069 | 11.1% | 0.013 | M1 = 6 |
| BC000450A | GNPAT-A | 0.045 | 12.9% | 0.009 | M3 = 11 | 0.223 | 11.1% | 0.054 | M2 = 6 |
| BC000770 | DIDO1 | 0.011 | 16.1% | 0.001 | M3 = 14 | 0.017 | 15.9% | 0.008 | M2 = 9 |
| BC002769A | C20orf43-A | 0.021 | 11.8% | 0.015 | M3 = 10 | 0.018 | 15.9% | 0.025 | M3 = 9 |
| BC004219A | AGPAT3-A | 0.016 | 14.0% | 0.005 | M3 = 12 | 0.000 | 23.8% | 0.001 | M3 = 14 |
| BC005029A | LEPREL1-A | 0.022 | 16.1% | 0.000 | M2 = 14 | 0.021 | 20.6% | 0.001 | M2 = 12 |
| BC005055A | FOXP1-A | 0.008 | 12.9% | 0.009 | M3 = 11 | 0.040 | 12.7% | 0.078 | M3 = 7 |
| BC007252 | COL2A1 | 0.007 | 14.0% | 0.001 | M2 = 12 | 0.030 | 12.7% | 0.029 | M2 = 7 |
| BC007581 | ALDH4A1 | 0.020 | 12.9% | 0.002 | M2 = 11 | 0.029 | 14.3% | 0.015 | M2 = 8 |
| BC007888 | EIF2S2 | 0.044 | 12.9% | 0.009 | M3 = 11 | 0.051 | 17.5% | 0.014 | M3 = 10 |
| BC009771 | BCCIP | 0.003 | 15.1% | 0.000 | M1 = 13 | 0.008 | 22.2% | 0.000 | M2 = 13 |
| BC009780 | SF3B3 | 0.018 | 15.1% | 0.002 | M3 = 13 | 0.010 | 20.6% | 0.004 | M3 = 12 |
| BC010032 | LASS2 | 0.044 | 11.8% | 0.015 | M3 = 10 | 0.054 | 17.5% | 0.014 | M3 = 10 |
| BC011519A | AGT-A | 0.007 | 12.9% | 0.009 | M3 = 11 | 0.014 | 11.1% | 0.013 | M1 = 6 |
| BC011603 | RELA | 0.015 | 14.0% | 0.005 | M3 = 12 | 0.018 | 12.7% | 0.006 | M1 = 7 |
| BC013116A | CLIPR-59-A | 0.026 | 12.9% | 0.002 | M2 = 11 | 0.060 | 14.3% | 0.015 | M2 = 8 |
| BC013424A | ARMC8-A | 0.011 | 11.8% | 0.015 | M3 = 10 | 0.028 | 12.7% | 0.029 | M2 = 7 |
| BC013567 | USP48 | 0.000 | 22.6% | 0.000 | M3 = 20 | 0.005 | 25.4% | 0.000 | M3 = 15 |
| BC013567A | USP48-A | 0.015 | 16.1% | 0.001 | M3 = 14 | 0.012 | 14.3% | 0.015 | M2 = 8 |
| BC015634 | COQ3 | 0.001 | 15.1% | 0.002 | M3 = 13 | 0.005 | 20.6% | 0.004 | M3 = 12 |
| BC015842A | EIF4A2-A | 0.042 | 16.1% | 0.001 | M3 = 14 | 0.079 | 17.5% | 0.014 | M3 = 10 |
| BC015944A | TIA1-A | 0.031 | 15.1% | 0.002 | M3 = 13 | 0.031 | 12.7% | 0.006 | M1 = 7 |
| BC016148 | ITM2B | 0.014 | 14.0% | 0.000 | M1 = 12 | 0.033 | 14.3% | 0.003 | M1 = 8 |
| BC017873 | FAM33A | 0.029 | 14.0% | 0.001 | M2 = 12 | 0.106 | 14.3% | 0.015 | M2 = 8 |
| BC018142A | CARD14-A | 0.053 | 12.9% | 0.009 | M3 = 11 | 0.493 | 11.1% | 0.131 | M3 = 6 |
| BC018722 | ASPSCR1 | 0.015 | 11.8% | 0.015 | M3 = 10 | 0.007 | 12.7% | 0.006 | M1 = 7 |
| BC020838 | CLDN20 | 0.006 | 11.8% | 0.015 | M3 = 10 | 0.002 | 15.9% | 0.008 | M2 = 9 |
| BC020843 | HAVCR2 | 0.034 | 11.8% | 0.015 | M3 = 10 | 0.012 | 19.0% | 0.007 | M3 = 11 |
| BC020843A | HAVCR2-A | 0.040 | 15.1% | 0.002 | M3 = 13 | 0.037 | 17.5% | 0.004 | M2 = 10 |
| BC021983 | NPM1 | 0.044 | 11.8% | 0.004 | M2 = 10 | 0.050 | 15.9% | 0.008 | M2 = 9 |
| BC022300A | ATP6V0A2-A | 0.012 | 11.8% | 0.001 | M1 = 10 | 0.035 | 12.7% | 0.006 | M1 = 7 |
| BC022454A | TRPM3-A | 0.013 | 11.8% | 0.004 | M2 = 10 | 0.038 | 12.7% | 0.029 | M2 = 7 |
| BC023006A | HSPCA-A | 0.048 | 14.0% | 0.005 | M3 = 12 | 0.034 | 15.9% | 0.001 | M1 = 9 |
| BC027889 | GJA4 | 0.034 | 14.0% | 0.005 | M3 = 12 | 0.087 | 11.1% | 0.054 | M2 = 6 |
| BC033035 | FLJ25758 | 0.028 | 11.8% | 0.015 | M3 = 10 | 0.035 | 12.7% | 0.029 | M2 = 7 |
| BC033529 | UPP2 | 0.017 | 16.1% | 0.001 | M3 = 14 | 0.032 | 17.5% | 0.014 | M3 = 10 |
| BC034468A | FLJ11171-A | 0.030 | 15.1% | 0.002 | M3 = 13 | 0.090 | 14.3% | 0.003 | M1 = 8 |
| BC034713A | DNAJC10-A | 0.022 | 15.1% | 0.001 | M2 = 13 | 0.022 | 20.6% | 0.001 | M2 = 12 |
| BC039832A | PPHLN1-A | 0.052 | 14.0% | 0.001 | M2 = 12 | 0.075 | 17.5% | 0.004 | M2 = 10 |
| BC040606A | WDR27-A | 0.002 | 15.1% | 0.001 | M2 = 13 | 0.017 | 17.5% | 0.004 | M2 = 10 |
| BC041157 | TBXAS1 | 0.016 | 12.9% | 0.009 | M3 = 11 | 0.035 | 12.7% | 0.006 | M1 = 7 |
| BC041158 | CYP4A11 | 0.040 | 12.9% | 0.002 | M2 = 11 | 0.106 | 12.7% | 0.029 | M2 = 7 |
| BC045532 | LSM8 | 0.002 | 11.8% | 0.015 | M3 = 10 | 0.004 | 14.3% | 0.045 | M3 = 8 |
| BC046567 | SLC37A3 | 0.030 | 11.8% | 0.015 | M3 = 10 | 0.058 | 15.9% | 0.025 | M3 = 9 |
| BC047703 | PLA1A | 0.037 | 14.0% | 0.005 | M3 = 12 | 0.068 | 14.3% | 0.015 | M2 = 8 |
| BC048251 | ZDHHC12 | 0.041 | 16.1% | 0.001 | M3 = 14 | 0.067 | 15.9% | 0.008 | M2 = 9 |
| BC050432 | STAU | 0.007 | 12.9% | 0.009 | M3 = 11 | 0.043 | 11.1% | 0.054 | M2 = 6 |
| BC050622 | REEP4/C8orf20 | 0.034 | 16.1% | 0.001 | M3 = 14 | 0.015 | 19.0% | 0.002 | M2 = 11 |
| BC050645A | BYSL-A | 0.015 | 14.0% | 0.005 | M3 = 12 | 0.047 | 11.1% | 0.013 | M1 = 6 |
| BC053533 | MTERFD2 | 0.016 | 11.8% | 0.015 | M3 = 10 | 0.107 | 14.3% | 0.045 | M3 = 8 |
| BC059364 | ZADH1 | 0.034 | 16.1% | 0.001 | M3 = 14 | 0.017 | 15.9% | 0.008 | M2 = 9 |
| BC064939 | VEZATIN | 0.029 | 11.8% | 0.015 | M3 = 10 | 0.058 | 17.5% | 0.014 | M3 = 10 |
| NM_000024 | ADRB2 | 0.002 | 12.9% | 0.009 | M3 = 11 | 0.009 | 22.2% | 0.002 | M3 = 13 |
| NM_000576A | IL1B-A | 0.045 | 11.8% | 0.004 | M2 = 10 | 0.069 | 12.7% | 0.006 | M1 = 7 |
| NM_001007098 | SCP2 | 0.007 | 16.1% | 0.001 | M3 = 14 | 0.022 | 17.5% | 0.004 | M2 = 10 |
| NM_001259 | CDK6 | 0.039 | 17.2% | 0.001 | M3 = 15 | 0.058 | 15.9% | 0.008 | M2 = 9 |
| NM_001671A | ASGR1-A | 0.020 | 15.1% | 0.002 | M3 = 13 | 0.033 | 14.3% | 0.015 | M2 = 8 |
| NM_001736A | C5R1-A | 0.016 | 12.9% | 0.009 | M3 = 11 | 0.050 | 14.3% | 0.015 | M2 = 8 |
| NM_002198 | IRF1 | 0.043 | 15.1% | 0.002 | M3 = 13 | 0.045 | 20.6% | 0.004 | M3 = 12 |
| NM_002982 | CCL2 | 0.052 | 11.8% | 0.015 | M3 = 10 | 0.101 | 12.7% | 0.006 | M1 = 7 |
| NM_003847 | PEX11A | 0.023 | 11.8% | 0.015 | M3 = 10 | 0.020 | 14.3% | 0.003 | M1 = 8 |
| NM_004001 | FCGR2B | 0.008 | 11.8% | 0.004 | M2 = 10 | 0.023 | 15.9% | 0.025 | M3 = 9 |
| NM_004401A | DFFA-A | 0.010 | 12.9% | 0.002 | M2 = 11 | 0.018 | 12.7% | 0.006 | M1 = 7 |
| NM_004402 | DFFB | 0.016 | 11.8% | 0.015 | M3 = 10 | 0.039 | 12.7% | 0.029 | M2 = 7 |
| NM_004431A | EPHA2-A | 0.002 | 16.1% | 0.001 | M3 = 14 | 0.018 | 11.1% | 0.013 | M1 = 6 |
| NM_004832A | GSTO1-A | 0.035 | 11.8% | 0.015 | M3 = 10 | 0.078 | 11.1% | 0.054 | M2 = 6 |
| NM_005290 | GPR15 | 0.040 | 16.1% | 0.000 | M2 = 14 | 0.124 | 19.0% | 0.002 | M2 = 11 |
| NM_005926 | MFAP1 | 0.001 | 11.8% | 0.015 | M3 = 10 | 0.001 | 27.0% | 0.000 | M2 = 16 |
| NM_006194 | PAX9 | 0.041 | 12.9% | 0.009 | M3 = 11 | 0.145 | 17.5% | 0.014 | M3 = 10 |
| NM_006253 | PRKAB1 | 0.019 | 14.0% | 0.005 | M3 = 12 | 0.027 | 12.7% | 0.006 | M1 = 7 |
| NM_006460A | HEXIM1-A | 0.003 | 16.1% | 0.001 | M3 = 14 | 0.017 | 15.9% | 0.008 | M2 = 9 |
| NM_012129 | CLDN12 | 0.002 | 16.1% | 0.001 | M3 = 14 | 0.004 | 15.9% | 0.008 | M2 = 9 |
| NM_014876 | KIAA0063 | 0.022 | 11.8% | 0.015 | M3 = 10 | 0.089 | 11.1% | 0.131 | M3 = 6 |
| NM_015381 | FAM19A5 | 0.024 | 11.8% | 0.015 | M3 = 10 | 0.006 | 15.9% | 0.008 | M2 = 9 |
| NM_015727A | TACR1-A | 0.051 | 11.8% | 0.015 | M3 = 10 | 0.206 | 14.3% | 0.045 | M3 = 8 |
| NM_016041 | DERL2 | 0.032 | 11.8% | 0.015 | M3 = 10 | 0.019 | 15.9% | 0.025 | M3 = 9 |
| NM_017727A | FLJ20254-A | 0.053 | 12.9% | 0.009 | M3 = 11 | 0.095 | 12.7% | 0.029 | M2 = 7 |
| NM_018246 | CCDC25 | 0.017 | 12.9% | 0.002 | M2 = 11 | 0.023 | 14.3% | 0.015 | M2 = 8 |
| NM_019103 | ZMAT5 | 0.046 | 11.8% | 0.015 | M3 = 10 | 0.166 | 11.1% | 0.131 | M3 = 6 |
| NM_030968 | C1QTNF1 | 0.020 | 12.9% | 0.000 | M1 = 11 | 0.054 | 15.9% | 0.025 | M3 = 9 |
| NM_031429 | RTBDN | 0.051 | 11.8% | 0.015 | M3 = 10 | 0.247 | 19.0% | 0.007 | M3 = 11 |
| NM_032676A | MGC10955-A | 0.009 | 14.0% | 0.001 | M2 = 12 | 0.013 | 15.9% | 0.008 | M2 = 9 |
| NM_033661A | WDR4-A | 0.008 | 11.8% | 0.004 | M2 = 10 | 0.020 | 11.1% | 0.013 | M1 = 6 |
| NM_080840A | PTPRA-A | 0.029 | 15.1% | 0.002 | M3 = 13 | 0.133 | 12.7% | 0.029 | M2 = 7 |
| NM_138778 | C9orf112 | 0.041 | 12.9% | 0.002 | M2 = 11 | 0.117 | 12.7% | 0.006 | M1 = 7 |
| NM_138784 | LOC116123 | 0.053 | 11.8% | 0.004 | M2 = 10 | 0.092 | 12.7% | 0.006 | M1 = 7 |
| NM_144594A | FLJ32942-A | 0.008 | 12.9% | 0.002 | M2 = 11 | 0.042 | 11.1% | 0.013 | M1 = 6 |
| NM_144628 | TBC1D20 | 0.015 | 12.9% | 0.002 | M2 = 11 | 0.037 | 15.9% | 0.025 | M3 = 9 |
| NM_145169A | SFT2D1-A | 0.001 | 14.0% | 0.000 | M1 = 12 | 0.016 | 11.1% | 0.013 | M1 = 6 |
| NM_145313A | RASGEF1A-A | 0.041 | 14.0% | 0.001 | M2 = 12 | 0.097 | 14.3% | 0.015 | M2 = 8 |
| NM_152452 | MGC18216 | 0.008 | 11.8% | 0.004 | M2 = 10 | 0.014 | 12.7% | 0.006 | M1 = 7 |
| NM_152789 | MGC40405 | 0.016 | 11.8% | 0.015 | M3 = 10 | 0.032 | 12.7% | 0.029 | M2 = 7 |
| NM_153229 | TMEM92 | 0.025 | 12.9% | 0.009 | M3 = 11 | 0.068 | 15.9% | 0.008 | M2 = 9 |
| NM_174903A | unknown-A | 0.044 | 12.9% | 0.009 | M3 = 11 | 0.068 | 14.3% | 0.045 | M3 = 8 |
| NM_174942A | GAS2L3-A | 0.009 | 14.0% | 0.005 | M3 = 12 | 0.123 | 12.7% | 0.029 | M2 = 7 |

Table 2 is a list of autoantigens that were bound more often by antibodies from sera from individuals one year prior to being diagnosed with lung adenocarcinoma than by antibodies from healthy individuals. The antigens in Table 2 are the same antigens listed in Table 1 and are identified according to Genbank ID number for the nucleotide sequence that encodes the antigens. It is understood that an antigen of Table 2 refers to a protein or fragments thereof that is encoded by the nucleotide sequence associated with the nucleotide ID number.

TABLE 2
Patients later diagnosed with lung adenocarcinoma vs.
healthy patients (same target antigens as Table 1)

| | | p-value | BEST | BEST | |
|---|---|---|---|---|---|
| | | t_Test | M-Stat | M-Stat | BEST |
| UI1 | SYMBOL | N, C, 1, 3 | Prev | p-value | M-Stat |
| BC000082A | ARS2-A | 0.082 | 25.0% | 0.026 | M2 = 7 |
| BC000450A | GNPAT-A | 0.044 | 34.4% | 0.011 | M3 = 10 |
| BC000770 | DIDO1 | 0.160 | 18.8% | 0.097 | M2 = 5 |
| BC002769A | C20orf43-A | 0.382 | 15.6% | 0.177 | M2 = 4 |
| BC004219A | AGPAT3-A | 0.390 | 25.0% | 0.073 | M3 = 7 |
| BC005029A | LEPREL1-A | 0.323 | 12.5% | 0.119 | M1 = 3 |
| BC005055A | FOXP1-A | 0.014 | 18.8% | 0.026 | M1 = 5 |
| BC007252 | COL2A1 | 0.055 | 25.0% | 0.073 | M3 = 7 |
| BC007581 | ALDH4A1 | 0.230 | 12.5% | 0.119 | M1 = 3 |
| BC007888 | EIF2S2 | 0.229 | 12.5% | 0.119 | M1 = 3 |
| BC009771 | BCCIP | 0.130 | 18.8% | 0.026 | M1 = 5 |
| BC009780 | SF3B3 | 0.411 | 12.5% | 0.306 | M2 = 3 |
| BC010032 | LASS2 | 0.334 | 12.5% | 0.119 | M1 = 3 |
| BC011519A | AGT-A | 0.151 | 15.6% | 0.177 | M2 = 4 |
| BC011603 | RELA | 0.277 | 15.6% | 0.177 | M2 = 4 |
| BC013116A | CLIPR-59-A | 0.081 | 18.8% | 0.026 | M1 = 5 |
| BC013424A | ARMC8-A | 0.190 | 12.5% | 0.306 | M2 = 3 |
| BC013567 | USP48 | 0.005 | 37.5% | 0.001 | M2 = 11 |
| BC013567A | USP48-A | 0.208 | 21.9% | 0.051 | M2 = 6 |
| BC015634 | COQ3 | 0.037 | 15.6% | 0.056 | M1 = 4 |
| BC015842A | EIF4A2-A | 0.054 | 21.9% | 0.051 | M2 = 6 |
| BC015944A | TIA1-A | 0.344 | 15.6% | 0.177 | M2 = 4 |
| BC016148 | ITM2B | 0.078 | 31.3% | 0.006 | M2 = 9 |
| BC017873 | FAM33A | 0.086 | 21.9% | 0.051 | M2 = 6 |
| BC018142A | CARD14-A | 0.041 | 21.9% | 0.012 | M1 = 6 |
| BC018722 | ASPSCR1 | 0.257 | 18.8% | 0.212 | M3 = 5 |
| BC020838 | CLDN20 | 0.348 | 15.6% | 0.177 | M2 = 4 |
| BC020843 | HAVCR2 | 0.414 | 12.5% | 0.500 | M3 = 3 |
| BC020843A | HAVCR2-A | 0.268 | 12.5% | 0.306 | M2 = 3 |
| BC021983 | NPM1 | 0.342 | 21.9% | 0.051 | M2 = 6 |
| BC022300A | ATP6V0A2-A | 0.104 | 25.0% | 0.073 | M3 = 7 |
| BC022454A | TRPM3-A | 0.089 | 12.5% | 0.119 | M1 = 3 |
| BC023006A | HSPCA-A | 0.458 | 12.5% | 0.500 | M3 = 3 |
| BC027889 | GJA4 | 0.113 | 18.8% | 0.026 | M1 = 5 |
| BC033035 | FLJ25758 | 0.275 | 12.5% | 0.306 | M2 = 3 |
| BC033529 | UPP2 | 0.144 | 25.0% | 0.005 | M1 = 7 |
| BC034468A | FLJ11171-A | 0.100 | 12.5% | 0.119 | M1 = 3 |
| BC034713A | DNAJC10-A | 0.295 | 12.5% | 0.119 | M1 = 3 |
| BC039832A | PPHLN1-A | 0.212 | 12.5% | 0.119 | M1 = 3 |
| BC040606A | WDR27-A | 0.016 | 21.9% | 0.051 | M2 = 6 |
| BC041157 | TBXAS1 | 0.108 | 12.5% | 0.306 | M2 = 3 |
| BC041158 | CYP4A11 | 0.107 | 18.8% | 0.097 | M2 = 5 |
| BC045532 | LSM8 | 0.107 | 15.6% | 0.177 | M2 = 4 |
| BC046567 | SLC37A3 | 0.163 | 25.0% | 0.073 | M3 = 7 |
| BC047703 | PLA1A | 0.180 | 18.8% | 0.097 | M2 = 5 |
| BC048251 | ZDHHC12 | 0.127 | 18.8% | 0.097 | M2 = 5 |
| BC050432 | STAU | 0.017 | 21.9% | 0.051 | M2 = 6 |
| BC050622 | REEP4/C8orf20 | 0.417 | 15.6% | 0.177 | M2 = 4 |
| BC050645A | BYSL-A | 0.084 | 18.8% | 0.097 | M2 = 5 |
| BC053533 | MTERFD2 | 0.035 | 28.1% | 0.002 | M1 = 8 |
| BC059364 | ZADH1 | 0.403 | 12.5% | 0.119 | M1 = 3 |
| BC064939 | VEZATIN | 0.083 | 18.8% | 0.097 | M2 = 5 |
| NM_000024 | ADRB2 | 0.053 | 31.3% | 0.006 | M2 = 9 |
| NM_000576A | IL1B-A | 0.148 | 25.0% | 0.073 | M3 = 7 |
| NM_001007098 | SCP2 | 0.088 | 28.1% | 0.013 | M2 = 8 |
| NM_001259 | CDK6 | 0.236 | 28.1% | 0.040 | M3 = 8 |
| NM_001671A | ASGR1-A | 0.245 | 18.8% | 0.097 | M2 = 5 |
| NM_001736A | C5R1-A | 0.068 | 12.5% | 0.119 | M1 = 3 |
| NM_002198 | IRF1 | 0.287 | 21.9% | 0.127 | M3 = 6 |
| NM_002982 | CCL2 | 0.225 | 18.8% | 0.212 | M3 = 5 |
| NM_003847 | PEX11A | 0.336 | 12.5% | 0.500 | M3 = 3 |
| NM_004001 | FCGR2B | 0.078 | 18.8% | 0.026 | M1 = 5 |
| NM_004401A | DFFA-A | 0.152 | 12.5% | 0.119 | M1 = 3 |
| NM_004402 | DFFB | 0.071 | 34.4% | 0.011 | M3 = 10 |
| NM_004431A | EPHA2-A | 0.020 | 18.8% | 0.026 | M1 = 5 |
| NM_004832A | GSTO1-A | 0.128 | 15.6% | 0.056 | M1 = 4 |
| NM_005290 | GPR15 | 0.098 | 15.6% | 0.056 | M1 = 4 |
| NM_005926 | MFAP1 | 0.201 | 28.1% | 0.040 | M3 = 8 |
| NM_006194 | PAX9 | 0.065 | 34.4% | 0.011 | M3 = 10 |
| NM_006253 | PRKAB1 | 0.179 | 15.6% | 0.177 | M2 = 4 |
| NM_006460A | HEXIM1-A | 0.038 | 21.9% | 0.051 | M2 = 6 |
| NM_012129 | CLDN12 | 0.085 | 37.5% | 0.001 | M2 = 11 |
| NM_014876 | KIAA0063 | 0.035 | 18.8% | 0.026 | M1 = 5 |
| NM_015381 | FAM19A5 | 0.455 | 15.6% | 0.335 | M3 = 4 |
| NM_015727A | TACR1-A | 0.060 | 12.5% | 0.119 | M1 = 3 |
| NM_016041 | DERL2 | 0.473 | 18.8% | 0.212 | M3 = 5 |
| NM_017727A | FLJ20254-A | 0.193 | 15.6% | 0.056 | M1 = 4 |
| NM_018246 | CCDC25 | 0.182 | 12.5% | 0.119 | M1 = 3 |
| NM_019103 | ZMAT5 | 0.106 | 18.8% | 0.026 | M1 = 5 |
| NM_030968 | C1QTNF1 | 0.067 | 25.0% | 0.005 | M1 = 7 |
| NM_031429 | RTBDN | 0.046 | 18.8% | 0.097 | M2 = 5 |
| NM_032676A | MGC10955-A | 0.062 | 28.1% | 0.013 | M2 = 8 |
| NM_033661A | WDR4-A | 0.075 | 15.6% | 0.056 | M1 = 4 |
| NM_080840A | PTPRA-A | 0.052 | 28.1% | 0.040 | M3 = 8 |
| NM_138778 | C9orf112 | 0.096 | 31.3% | 0.021 | M3 = 9 |
| NM_138784 | LOC116123 | 0.134 | 15.6% | 0.177 | M2 = 4 |
| NM_144594A | FLJ32942-A | 0.018 | 31.3% | 0.021 | M3 = 9 |
| NM_144628 | TBC1D20 | 0.146 | 21.9% | 0.012 | M1 = 6 |
| NM_145169A | SFT2D1-A | 0.010 | 31.3% | 0.021 | M3 = 9 |
| NM_145313A | RASGEF1A-A | 0.132 | 15.6% | 0.056 | M1 = 4 |
| NM_152452 | MGC18216 | 0.136 | 18.8% | 0.097 | M2 = 5 |
| NM_152789 | MGC40405 | 0.156 | 15.6% | 0.177 | M2 = 4 |
| NM_153229 | TMEM92 | 0.102 | 21.9% | 0.127 | M3 = 6 |
| NM_174903A | unknown-A | 0.123 | 15.6% | 0.056 | M1 = 4 |
| NM_174942A | GAS2L3-A | 0.019 | 34.4% | 0.011 | M3 = 10 |

Tables 3 and 4 list target antigens that were upregulated (Table 3) or downregulated (Table 4) in patients diagnosed with lung adenocarcinoma compared to normal individuals. It is understood that an antigen of Table 3 or Table 4 refers to a protein or fragments thereof that is encoded by the nucleotide sequence associated with the nucleotide ID number. The suffix (A) in the UI1 and Symbol columns denotes detection with anti-human IgA secondary antibody. Column UI1 reports the GenBank Accession number for each antigen, and column UI2 reports the IVGN Ultimate ORF Collection ID number. The power ratio is a simple division of normalized value of third highest Cancer Sample by normalized value of third highest Normal Sample (or Normal by Cancer). Prevalence and M-Statistic p-value are calculated as described herein.

TABLE 3
Target antigens that were upregulated in patients diagnosed with lung
adenocarcinoma compared to normal individuals.

| | | | t-test | RATIO | BEST | BEST | |
|---|---|---|---|---|---|---|---|
| 248 UP in LAC | | | p-value | 3-CA + | M-Stat | M-Stat | BEST |
| UI1 | UI2 | SYMBOL | N, C, 1, 3 | 3-NO | Prev | p-value | M-Stat |
| BC000082A | IOH4680 | ARS2-A | 0.028 | 4.62 | 16.1% | 0.001 | M3 = 14 |
| BC000450A | IOH3506 | GNPAT-A | 0.045 | 2.04 | 12.9% | 0.009 | M3 = 11 |
| BC000526 | IOH3637 | TMEM98 | 0.036 | 2.02 | 7.5% | 0.014 | M1 = 6 |
| BC000742A | IOH4675 | GPX1-A | 0.026 | 1.88 | 7.5% | 0.014 | M1 = 6 |
| BC001017A | IOH4237 | PDLIM3-A | 0.015 | 2.14 | 11.8% | 0.004 | M2 = 10 |
| BC001152 | IOH4425 | GAS7 | 0.039 | 2.34 | 9.7% | 0.048 | M3 = 8 |
| BC001152A | IOH4425 | GAS7-A | 0.052 | 2.17 | 7.5% | 0.014 | M1 = 6 |
| BC001772A | IOH4925 | QARS-A | 0.041 | 3.22 | 7.5% | 0.014 | M1 = 6 |
| BC001868A | IOH4965 | ZNF44-A | 0.039 | 2.19 | 11.8% | 0.015 | M3 = 10 |
| BC002509A | IOH3968 | PHF23-A | 0.030 | 1.82 | 7.5% | 0.014 | M1 = 6 |
| BC002755 | NotAvailable | MKNK1 | 0.025 | 2.30 | 9.7% | 0.017 | M2 = 8 |
| BC002769A | IOH5310 | C20orf43-A | 0.021 | 3.56 | 11.8% | 0.015 | M3 = 10 |
| BC002880A | IOH5687 | CARS-A | 0.019 | 2.76 | 7.5% | 0.014 | M1 = 6 |
| BC002906A | IOH5684 | UCK2-A | 0.041 | 3.30 | 9.7% | 0.048 | M3 = 8 |
| BC003643 | IOH5184 | DHDDS | 0.019 | 1.87 | 10.8% | 0.009 | M2 = 9 |
| BC004219A | IOH5155 | AGPAT3-A | 0.016 | 2.14 | 14.0% | 0.005 | M3 = 12 |
| BC005029A | IOH6657 | LEPREL1-A | 0.022 | 2.67 | 16.1% | 0.000 | M2 = 14 |
| BC005055A | IOH6528 | FOXP1-A | 0.008 | 3.53 | 12.9% | 0.009 | M3 = 11 |
| BC005187A | IOH7207 | UFC1-A | 0.050 | 3.72 | 10.8% | 0.028 | M3 = 9 |
| BC007252 | IOH6830 | COL2A1 | 0.007 | 2.47 | 14.0% | 0.001 | M2 = 12 |
| BC007347A | IOH5863 | CHD2-A | 0.021 | 3.93 | 12.9% | 0.002 | M2 = 11 |
| BC007407A | IOH5830 | NMB-A | 0.046 | 2.46 | 11.8% | 0.015 | M3 = 10 |
| BC007581 | IOH6857 | ALDH4A1 | 0.020 | 3.05 | 12.9% | 0.002 | M2 = 11 |
| BC007888 | IOH29315 | EIF2S2 | 0.044 | 2.07 | 12.9% | 0.009 | M3 = 11 |
| BC008094A | IOH3305 | RGR-A | 0.022 | 2.65 | 9.7% | 0.017 | M2 = 8 |
| BC008141A | IOH10197 | TREX2-A | 0.040 | 1.89 | 11.8% | 0.004 | M2 = 10 |
| BC008302A | NotAvailable | ALS2CR2-A | 0.040 | 2.41 | 10.8% | 0.009 | M2 = 9 |
| BC008365A | IOH5958 | unknown-A | 0.046 | 2.70 | 7.5% | 0.014 | M1 = 6 |
| BC009106A | IOH10263 | LZTR2-A | 0.014 | 2.47 | 8.6% | 0.007 | M1 = 7 |
| BC009249A | IOH12481 | KPTN-A | 0.007 | 3.54 | 7.5% | 0.014 | M1 = 6 |
| BC009251 | IOH27776 | FDFT1 | 0.018 | 2.17 | 8.6% | 0.007 | M1 = 7 |
| BC009650 | IOH11334 | SCC-112 | 0.029 | 2.69 | 6.5% | 0.029 | M1 = 5 |
| BC009674 | IOH9894 | YIPF1 | 0.019 | 2.28 | 10.8% | 0.028 | M3 = 9 |
| BC009771 | IOH12849 | BCCIP | 0.003 | 4.17 | 15.1% | 0.000 | M1 = 13 |
| BC009877A | IOH12614 | P2RY11-A | 0.012 | 2.60 | 6.5% | 0.029 | M1 = 5 |
| BC010640A | IOH9738 | STK3-A | 0.007 | 1.81 | 10.8% | 0.002 | M1 = 9 |
| BC010704 | IOH9730 | SH2B | 0.038 | 2.49 | 9.7% | 0.017 | M2 = 8 |
| BC010887A | IOH27768 | REC8L1-A | 0.043 | 3.16 | 10.8% | 0.009 | M2 = 9 |
| BC011519A | IOH9674 | AGT-A | 0.007 | 1.98 | 12.9% | 0.009 | M3 = 11 |
| BC011563A | IOH12467 | LAT-A | 0.013 | 3.62 | 14.0% | 0.005 | M3 = 12 |
| BC011578A | IOH12646 | CD276-A | 0.043 | 3.49 | 8.6% | 0.007 | M1 = 7 |
| BC011603 | IOH13717 | RELA | 0.015 | 2.76 | 14.0% | 0.005 | M3 = 12 |
| BC011603A | IOH13717 | RELA-A | 0.023 | 3.21 | 6.5% | 0.029 | M1 = 5 |
| BC012547A | IOH14261 | EIF4A2-A | 0.022 | 5.40 | 9.7% | 0.017 | M2 = 8 |
| BC012566 | IOH27796 | NPM1 | 0.018 | 2.36 | 11.8% | 0.004 | M2 = 10 |
| BC012984A | IOH13016 | ALS2CR19-A | 0.030 | 2.92 | 9.7% | 0.003 | M1 = 8 |
| BC013103 | IOH22117 | RHBDL2 | 0.047 | 1.87 | 7.5% | 0.014 | M1 = 6 |
| BC013116A | IOH28618 | CLIPR-59-A | 0.026 | 5.47 | 12.9% | 0.002 | M2 = 11 |
| BC013424A | IOH9880 | ARMC8-A | 0.011 | 3.07 | 11.8% | 0.015 | M3 = 10 |
| BC013567A | IOH9922 | USP48-A | 0.015 | 3.11 | 16.1% | 0.001 | M3 = 14 |
| BC013768A | IOH11543 | PCCB-A | 0.014 | 3.75 | 8.6% | 0.031 | M2 = 7 |
| BC013791A | IOH22123 | PCP4-A | 0.016 | 3.33 | 7.5% | 0.014 | M1 = 6 |
| BC013905A | IOH14523 | unknown-A | 0.041 | 2.70 | 6.5% | 0.029 | M1 = 5 |
| BC014057A | IOH14544 | DHRS1-A | 0.030 | 2.10 | 6.5% | 0.029 | M1 = 5 |
| BC014095 | IOH14089 | RELA | 0.018 | 1.85 | 6.5% | 0.029 | M1 = 5 |
| BC014095A | IOH14089 | RELA-A | 0.015 | 2.49 | 11.8% | 0.015 | M3 = 10 |
| BC014665A | IOH14248 | ADH5-A | 0.015 | 3.68 | 8.6% | 0.007 | M1 = 7 |
| BC014891 | IOH10029 | KIAA0701 | 0.036 | 1.94 | 9.7% | 0.003 | M1 = 8 |
| BC015634 | IOH14765 | COQ3 | 0.001 | 4.15 | 15.1% | 0.002 | M3 = 13 |
| BC015842A | IOH23072 | EIF4A2-A | 0.042 | 5.23 | 16.1% | 0.001 | M3 = 14 |
| BC015878A | IOH23080 | NAT2-A | 0.031 | 3.34 | 7.5% | 0.014 | M1 = 6 |
| BC015944A | IOH10042 | TIA1-A | 0.031 | 2.98 | 15.1% | 0.002 | M3 = 13 |
| BC016148 | IOH10092 | ITM2B | 0.014 | 1.84 | 14.0% | 0.000 | M1 = 12 |
| BC016295A | IOH10091 | EIF4A2-A | 0.048 | 2.34 | 10.8% | 0.028 | M3 = 9 |
| BC016640A | IOH21476 | SSX5-A | 0.025 | 3.79 | 14.0% | 0.005 | M3 = 12 |
| BC017025A | IOH9960 | PTMS-A | 0.051 | 2.09 | 6.5% | 0.029 | M1 = 5 |
| BC017046A | IOH11667 | ANXA6-A | 0.023 | 3.62 | 6.5% | 0.029 | M1 = 5 |
| BC017314A | IOH14156 | ETS1-A | 0.033 | 2.78 | 7.5% | 0.014 | M1 = 6 |
| BC017715 | NotAvailable | MAP3K7 | 0.009 | 3.27 | 7.5% | 0.014 | M1 = 6 |
| BC018142A | IOH10362 | CARD14-A | 0.053 | 4.06 | 12.9% | 0.009 | M3 = 11 |
| BC018404A | IOH21454 | FGF21-A | 0.039 | 2.01 | 10.8% | 0.028 | M3 = 9 |
| BC018722 | IOH13970 | ASPSCR1 | 0.015 | 2.82 | 11.8% | 0.015 | M3 = 10 |
| BC020838 | IOH13454 | CLDN20 | 0.006 | 2.31 | 11.8% | 0.015 | M3 = 10 |
| BC020843A | IOH12055 | HAVCR2-A | 0.040 | 2.55 | 15.1% | 0.002 | M3 = 13 |
| BC021983 | IOH27884 | NPM1 | 0.044 | 1.91 | 11.8% | 0.004 | M2 = 10 |
| BC021988 | IOH14635 | NDFIP2 | 0.039 | 1.90 | 6.5% | 0.029 | M1 = 5 |
| BC022300A | IOH12946 | ATP6V0A2-A | 0.012 | 2.35 | 11.8% | 0.001 | M1 = 10 |
| BC022302 | IOH14393 | CMAH | 0.033 | 1.84 | 9.7% | 0.003 | M1 = 8 |
| BC022454A | IOH10977 | TRPM3-A | 0.013 | 2.99 | 11.8% | 0.004 | M2 = 10 |
| BC023006A | IOH22358 | HSPCA-A | 0.048 | 3.15 | 14.0% | 0.005 | M3 = 12 |
| BC024272A | IOH12488 | CD74-A | 0.044 | 1.91 | 8.6% | 0.031 | M2 = 7 |
| BC025761 | IOH11223 | CYP2W1 | 0.038 | 2.24 | 11.8% | 0.004 | M2 = 10 |
| BC027866A | IOH12025 | ST8SIA4-A | 0.006 | 1.85 | 8.6% | 0.031 | M2 = 7 |
| BC028739A | IOH11395 | KCNV1-A | 0.032 | 2.41 | 9.7% | 0.048 | M3 = 8 |
| BC028917A | IOH22195 | ZC3HC1-A | 0.028 | 2.79 | 8.6% | 0.007 | M1 = 7 |
| BC030020A | IOH22410 | DDX55-A | 0.023 | 3.58 | 7.5% | 0.014 | M1 = 6 |
| BC032108 | IOH23020 | VPS52 | 0.025 | 3.26 | 9.7% | 0.003 | M1 = 8 |
| BC032578A | IOH21927 | WDR19-A | 0.051 | 1.85 | 9.7% | 0.048 | M3 = 8 |
| BC032864A | IOH28650 | GLRA2-A | 0.032 | 4.08 | 6.5% | 0.029 | M1 = 5 |
| BC033035 | IOH22449 | FLJ25758 | 0.028 | 3.56 | 11.8% | 0.015 | M3 = 10 |
| BC033196A | IOH23248 | MGC45840-A | 0.047 | 4.80 | 10.8% | 0.009 | M2 = 9 |
| BC033529 | IOH27634 | UPP2 | 0.017 | 2.52 | 16.1% | 0.001 | M3 = 14 |
| BC034275A | IOH21410 | ASPHD1-A | 0.021 | 1.82 | 6.5% | 0.029 | M1 = 5 |
| BC034468A | IOH22282 | FLJ11171-A | 0.030 | 1.92 | 15.1% | 0.002 | M3 = 13 |
| BC034483A | IOH22297 | HSPA1L-A | 0.027 | 1.95 | 9.7% | 0.048 | M3 = 8 |
| BC034713A | IOH22181 | DNAJC10-A | 0.022 | 2.54 | 15.1% | 0.001 | M2 = 13 |
| BC035198 | IOH28613 | PKM2 | 0.039 | 2.21 | 8.6% | 0.007 | M1 = 7 |
| BC035573A | IOH27592 | TCEAL8-A | 0.014 | 2.49 | 9.7% | 0.003 | M1 = 8 |
| BC036242 | IOH27520 | CPNE3 | 0.013 | 2.00 | 10.8% | 0.028 | M3 = 9 |
| BC036651A | IOH28634 | PTK2B-A | 0.023 | 3.17 | 10.8% | 0.009 | M2 = 9 |
| BC037278A | IOH27106 | TMEM104-A | 0.032 | 2.78 | 8.6% | 0.007 | M1 = 7 |
| BC039577A | IOH26185 | PNMA1-A | 0.037 | 2.35 | 6.5% | 0.029 | M1 = 5 |
| BC039832A | IOH26274 | PPHLN1-A | 0.052 | 1.87 | 14.0% | 0.001 | M2 = 12 |
| BC040606A | IOH27734 | WDR27-A | 0.002 | 3.63 | 15.1% | 0.001 | M2 = 13 |
| BC042820 | IOH26481 | PSMA8 | 0.013 | 2.18 | 7.5% | 0.014 | M1 = 6 |
| BC043391A | IOH26414 | TAF7L-A | 0.035 | 3.38 | 7.5% | 0.014 | M1 = 6 |
| BC050432A | IOH26694 | STAU-A | 0.051 | 2.12 | 8.6% | 0.007 | M1 = 7 |
| BC050622 | IOH27083 | REEP4/C8orf20 | 0.034 | 2.11 | 16.1% | 0.001 | M3 = 14 |
| BC050645A | IOH27032 | BYSL-A | 0.015 | 3.48 | 14.0% | 0.005 | M3 = 12 |
| BC050688 | IOH27002 | RPSA | 0.034 | 2.58 | 9.7% | 0.048 | M3 = 8 |
| BC051374 | IOH28132 | SCN5A | 0.051 | 1.75 | 6.5% | 0.029 | M1 = 5 |
| BC051874A | IOH27064 | CROT-A | 0.014 | 2.08 | 10.8% | 0.028 | M3 = 9 |
| BC051911 | IOH27047 | C13orf24 | 0.035 | 2.09 | 9.7% | 0.048 | M3 = 8 |
| BC052591A | IOH29389 | PPT2-A | 0.024 | 2.93 | 7.5% | 0.014 | M1 = 6 |
| BC052803A | IOH29397 | FOXP4-A | 0.040 | 3.11 | 6.5% | 0.029 | M1 = 5 |
| BC053610A | IOH28998 | PRKAB2-A | 0.010 | 2.30 | 10.8% | 0.009 | M2 = 9 |
| BC058906 | IOH29100 | ADCK1 | 0.001 | 1.78 | 16.1% | 0.000 | M2 = 14 |
| BC063463 | IOH39865 | COQ3 | 0.005 | 1.87 | 9.7% | 0.017 | M2 = 8 |
| BC067299 | IOH40040 | MDM4 | 0.009 | 4.08 | 10.8% | 0.009 | M2 = 9 |
| BC068094A | IOH40788 | SH3TC1-A | 0.046 | 3.40 | 9.7% | 0.048 | M3 = 8 |
| NM_000024 | IOH29873 | ADRB2 | 0.002 | 2.29 | 12.9% | 0.009 | M3 = 11 |
| NM_000358A | IOH5136 | TGFBI-A | 0.029 | 2.34 | 8.6% | 0.007 | M1 = 7 |
| NM_000398A | IOH3023 | CYB5R3-A | 0.042 | 1.92 | 7.5% | 0.014 | M1 = 6 |
| NM_000576A | IOH9821 | IL1B-A | 0.045 | 2.44 | 11.8% | 0.004 | M2 = 10 |
| NM_000858A | IOH6313 | GUK1-A | 0.048 | 2.24 | 10.8% | 0.028 | M3 = 9 |
| NM_001154A | IOH5099 | ANXA5-A | 0.024 | 2.32 | 6.5% | 0.029 | M1 = 5 |
| NM_001239A | IOH7258 | CCNH-A | 0.015 | 3.48 | 8.6% | 0.007 | M1 = 7 |
| NM_001259 | IOH28111 | CDK6 | 0.039 | 1.97 | 17.2% | 0.001 | M3 = 15 |
| NM_001450 | IOH14447 | FHL2 | 0.042 | 1.81 | 11.8% | 0.015 | M3 = 10 |
| NM_001549A | IOH3948 | IFIT3-A | 0.012 | 1.85 | 8.6% | 0.031 | M2 = 7 |
| NM_001637A | IOH10670 | AOAH-A | 0.048 | 2.46 | 10.8% | 0.028 | M3 = 9 |
| NM_001671A | IOH23108 | ASGR1-A | 0.020 | 2.72 | 15.1% | 0.002 | M3 = 13 |
| NM_001688 | IOH7382 | ATP5F1 | 0.044 | 2.06 | 11.8% | 0.015 | M3 = 10 |
| NM_001736A | IOH3294 | C5R1-A | 0.016 | 3.14 | 12.9% | 0.009 | M3 = 11 |
| NM_002055A | IOH9657 | GFAP-A | 0.048 | 2.13 | 10.8% | 0.028 | M3 = 9 |
| NM_002316A | IOH34707 | LMX1B-A | 0.044 | 2.79 | 7.5% | 0.014 | M1 = 6 |
| NM_002435 | IOH26367 | MPI | 0.025 | 6.14 | 8.6% | 0.031 | M2 = 7 |
| NM_002540A | IOH40890 | ODF2-A | 0.050 | 1.83 | 6.5% | 0.029 | M1 = 5 |
| NM_002752A | NotAvailable | MAPK9-A | 0.052 | 2.47 | 6.5% | 0.029 | M1 = 5 |
| NM_002795A | IOH14510 | PSMB3-A | 0.041 | 2.17 | 10.8% | 0.009 | M2 = 9 |
| NM_002904 | IOH14621 | RDBP | 0.045 | 2.68 | 6.5% | 0.029 | M1 = 5 |
| NM_003010A | NotAvailable | MAP2K4-A | 0.007 | 2.25 | 10.8% | 0.002 | M1 = 9 |
| NM_003315A | IOH14566 | DNAJC7-A | 0.024 | 2.32 | 11.8% | 0.004 | M2 = 10 |
| NM_003827A | IOH4804 | NAPA-A | 0.028 | 10.41 | 9.7% | 0.048 | M3 = 8 |
| NM_003831 | IOH20968 | RIOK3 | 0.038 | 2.63 | 6.5% | 0.029 | M1 = 5 |
| NM_003845 | IOH21591 | DYRK4 | 0.003 | 1.77 | 6.5% | 0.029 | M1 = 5 |
| NM_003878A | IOH25778 | GGH-A | 0.047 | 2.32 | 12.9% | 0.009 | M3 = 11 |
| NM_004311 | IOH22951 | ARL3 | 0.012 | 1.84 | 10.8% | 0.028 | M3 = 9 |
| NM_004401A | IOH4671 | DFFA-A | 0.010 | 4.10 | 12.9% | 0.002 | M2 = 11 |
| NM_004431A | NotAvailable | EPHA2-A | 0.002 | 2.98 | 16.1% | 0.001 | M3 = 14 |
| NM_004443A | NotAvailable | EPHB3-A | 0.032 | 2.25 | 11.8% | 0.015 | M3 = 10 |
| NM_004492A | IOH6006 | GTF2A2-A | 0.045 | 2.54 | 6.5% | 0.029 | M1 = 5 |
| NM_004586A | NotAvailable | RPS6KA3-A | 0.008 | 2.55 | 8.6% | 0.007 | M1 = 7 |
| NM_004832A | IOH4381 | GSTO1-A | 0.035 | 2.49 | 11.8% | 0.015 | M3 = 10 |
| NM_004844A | IOH13842 | SH3BP5-A | 0.033 | 4.04 | 11.8% | 0.015 | M3 = 10 |
| NM_004906 | IOH40770 | WTAP | 0.021 | 1.87 | 9.7% | 0.003 | M1 = 8 |
| NM_004906A | IOH40770 | WTAP-A | 0.020 | 3.19 | 10.8% | 0.002 | M1 = 9 |
| NM_004906A | IOH40770 | WTAP-A | 0.012 | 2.05 | 10.8% | 0.028 | M3 = 9 |
| NM_005086A | IOH1877 | SSPN-A | 0.028 | 2.49 | 10.8% | 0.002 | M1 = 9 |
| NM_005309 | IOH9641 | GPT | 0.050 | 2.52 | 10.8% | 0.009 | M2 = 9 |
| NM_005666A | IOH13657 | CFHL2-A | 0.038 | 3.89 | 8.6% | 0.031 | M2 = 7 |
| NM_005723A | IOH9899 | TSPAN5-A | 0.023 | 2.23 | 8.6% | 0.007 | M1 = 7 |
| NM_005832A | IOH12198 | KCNMB2-A | 0.045 | 2.11 | 9.7% | 0.017 | M2 = 8 |
| NM_005923A | NotAvailable | MAP3K5-A | 0.027 | 2.17 | 14.0% | 0.001 | M2 = 12 |
| NM_006460A | IOH5964 | HEXIM1-A | 0.003 | 4.32 | 16.1% | 0.001 | M3 = 14 |
| NM_006578A | IOH14466 | GNB5-A | 0.034 | 3.29 | 6.5% | 0.029 | M1 = 5 |
| NM_006622A | NotAvailable | PLK2-A | 0.042 | 2.15 | 6.5% | 0.029 | M1 = 5 |
| NM_006807A | IOH4111 | CBX1-A | 0.052 | 1.87 | 10.8% | 0.028 | M3 = 9 |
| NM_006977A | IOH26026 | ZBTB25-A | 0.041 | 3.14 | 7.5% | 0.014 | M1 = 6 |
| NM_007108A | IOH14150 | TCEB2-A | 0.052 | 2.03 | 6.5% | 0.029 | M1 = 5 |
| NM_007189A | IOH3871 | ABCF2-A | 0.044 | 3.13 | 9.7% | 0.017 | M2 = 8 |
| NM_012097 | IOH3083 | ARL5 | 0.040 | 2.31 | 10.8% | 0.028 | M3 = 9 |
| NM_013290A | IOH6212 | TBPIP-A | 0.031 | 2.52 | 9.7% | 0.017 | M2 = 8 |
| NM_014234A | IOH3322 | HSD17B8-A | 0.045 | 2.59 | 9.7% | 0.017 | M2 = 8 |
| NM_014241A | IOH12805 | PTPLA-A | 0.008 | 1.84 | 10.8% | 0.028 | M3 = 9 |
| NM_014496A | NotAvailable | RPS6KA6-A | 0.017 | 2.60 | 7.5% | 0.014 | M1 = 6 |
| NM_014519 | IOH40635 | ZNF232 | 0.005 | 2.14 | 8.6% | 0.007 | M1 = 7 |
| NM_014876 | IOH10136 | KIAA0063 | 0.022 | 2.76 | 11.8% | 0.015 | M3 = 10 |
| NM_016103 | IOH5421 | SARA2 | 0.029 | 2.77 | 10.8% | 0.028 | M3 = 9 |
| NM_016282A | IOH11046 | AK3-A | 0.015 | 4.20 | 7.5% | 0.014 | M1 = 6 |
| NM_016301A | IOH7464 | ATPBD1C-A | 0.027 | 1.76 | 9.7% | 0.048 | M3 = 8 |
| NM_016401A | IOH3734 | HSPC138-A | 0.027 | 2.54 | 9.7% | 0.003 | M1 = 8 |
| NM_016940A | IOH12821 | C21orf6-A | 0.034 | 1.81 | 8.6% | 0.031 | M2 = 7 |
| NM_017444 | IOH10035 | CHRAC1 | 0.026 | 1.87 | 7.5% | 0.014 | M1 = 6 |
| NM_017727A | IOH4312 | FLJ20254-A | 0.053 | 2.11 | 12.9% | 0.009 | M3 = 11 |
| NM_017867A | IOH12106 | FLJ20534-A | 0.052 | 2.69 | 9.7% | 0.017 | M2 = 8 |
| NM_018246 | IOH40864 | CCDC25 | 0.017 | 9.18 | 12.9% | 0.002 | M2 = 11 |
| NM_018246A | IOH40864 | CCDC25-A | 0.019 | 2.72 | 9.7% | 0.017 | M2 = 8 |
| NM_018291A | IOH14716 | FLJ10986-A | 0.038 | 3.02 | 14.0% | 0.005 | M3 = 12 |
| NM_018650A | NotAvailable | MARK1-A | 0.040 | 1.78 | 12.9% | 0.009 | M3 = 11 |
| NM_019613A | IOH6706 | WDR45L-A | 0.018 | 2.71 | 7.5% | 0.014 | M1 = 6 |
| NM_020070 | IOH10617 | IGLL1 | 0.039 | 2.00 | 11.8% | 0.001 | M1 = 10 |
| NM_020384A | IOH12584 | CLDN2-A | 0.007 | 2.02 | 7.5% | 0.014 | M1 = 6 |
| NM_020992A | IOH2948 | PDLIM1-A | 0.041 | 2.38 | 10.8% | 0.028 | M3 = 9 |
| NM_021105A | IOH13995 | PLSCR1-A | 0.046 | 2.36 | 6.5% | 0.029 | M1 = 5 |
| NM_021123A | IOH27363 | GAGE7-A | 0.039 | 3.19 | 10.8% | 0.028 | M3 = 9 |
| NM_021969A | IOH22604 | NR0B2-A | 0.036 | 1.83 | 7.5% | 0.014 | M1 = 6 |
| NM_022048A | IOH21026 | CSNK1G1-A | 0.020 | 2.17 | 8.6% | 0.007 | M1 = 7 |
| NM_022133A | IOH21896 | SNX16-A | 0.044 | 2.82 | 9.7% | 0.048 | M3 = 8 |
| NM_022788A | IOH12543 | P2RY12-A | 0.044 | 2.39 | 6.5% | 0.029 | M1 = 5 |
| NM_022972A | NotAvailable | FGFR2-A | 0.046 | 2.95 | 10.8% | 0.028 | M3 = 9 |
| NM_024056A | IOH3247 | MGC5576-A | 0.037 | 2.25 | 6.5% | 0.029 | M1 = 5 |
| NM_024060A | IOH21405 | AHNAK-A | 0.011 | 1.88 | 12.9% | 0.009 | M3 = 11 |
| NM_024299A | IOH4080 | C20orf149-A | 0.014 | 3.56 | 9.7% | 0.048 | M3 = 8 |
| NM_024331A | IOH3905 | C20orf121-A | 0.037 | 4.90 | 10.8% | 0.009 | M2 = 9 |
| NM_024901A | IOH6373 | DENND2D-A | 0.023 | 2.24 | 8.6% | 0.007 | M1 = 7 |
| NM_025161A | IOH14763 | C17orf70-A | 0.039 | 2.23 | 8.6% | 0.031 | M2 = 7 |
| NM_030815A | IOH12796 | PDRG1-A | 0.037 | 2.52 | 6.5% | 0.029 | M1 = 5 |
| NM_030968 | IOH13365 | C1QTNF1 | 0.020 | 2.09 | 12.9% | 0.000 | M1 = 11 |
| NM_031896A | IOH39520 | CACNG7-A | 0.045 | 1.89 | 10.8% | 0.028 | M3 = 9 |
| NM_032047A | IOH27467 | B3GNT5-A | 0.026 | 1.81 | 6.5% | 0.029 | M1 = 5 |
| NM_032509A | IOH26995 | RBM13-A | 0.044 | 3.06 | 7.5% | 0.014 | M1 = 6 |
| NM_032676A | IOH5559 | MGC10955-A | 0.009 | 2.66 | 14.0% | 0.001 | M2 = 12 |
| NM_033661A | IOH6391 | WDR4-A | 0.008 | 3.03 | 11.8% | 0.004 | M2 = 10 |
| NM_080590A | IOH13252 | CAPS-A | 0.020 | 2.04 | 10.8% | 0.028 | M3 = 9 |
| NM_080658A | IOH3352 | ACY3-A | 0.021 | 1.87 | 11.8% | 0.004 | M2 = 10 |
| NM_080675A | IOH10614 | SPAG4L-A | 0.019 | 3.58 | 11.8% | 0.001 | M1 = 10 |
| NM_080840A | IOH11085 | PTPRA-A | 0.029 | 2.40 | 15.1% | 0.002 | M3 = 13 |
| NM_133484 | IOH2979 | TANK | 0.042 | 4.82 | 10.8% | 0.009 | M2 = 9 |
| NM_134442A | IOH9797 | CREB1-A | 0.024 | 2.95 | 7.5% | 0.014 | M1 = 6 |
| NM_138278A | IOH11810 | BNIPL-A | 0.025 | 2.79 | 9.7% | 0.017 | M2 = 8 |
| NM_138419 | IOH22975 | FAM54A | 0.004 | 2.18 | 8.6% | 0.007 | M1 = 7 |
| NM_138446A | IOH13334 | C7orf30-A | 0.025 | 1.78 | 8.6% | 0.007 | M1 = 7 |
| NM_138461A | IOH21483 | TM4SF19-A | 0.020 | 2.18 | 8.6% | 0.031 | M2 = 7 |
| NM_144594A | IOH10942 | FLJ32942-A | 0.008 | 3.63 | 12.9% | 0.002 | M2 = 11 |
| NM_144628 | IOH12982 | TBC1D20 | 0.015 | 1.80 | 12.9% | 0.002 | M2 = 11 |
| NM_144676A | IOH13348 | TMED6-A | 0.021 | 2.12 | 7.5% | 0.014 | M1 = 6 |
| NM_145041A | IOH13199 | MGC20235-A | 0.007 | 3.48 | 10.8% | 0.028 | M3 = 9 |
| NM_145047A | IOH10837 | C1orf102-A | 0.029 | 2.59 | 10.8% | 0.009 | M2 = 9 |
| NM_145050A | IOH12883 | CCDC26-A | 0.025 | 2.67 | 7.5% | 0.014 | M1 = 6 |
| NM_145169A | IOH14571 | SFT2D1-A | 0.001 | 2.43 | 14.0% | 0.000 | M1 = 12 |
| NM_145313A | IOH10825 | RASGEF1A-A | 0.041 | 2.12 | 14.0% | 0.001 | M2 = 12 |
| NM_145793A | IOH13552 | GFRA1-A | 0.041 | 3.26 | 6.5% | 0.029 | M1 = 5 |
| NM_148957A | IOH26603 | TNFRSF19-A | 0.050 | 3.31 | 7.5% | 0.014 | M1 = 6 |
| NM_152688 | IOH22222 | KHDRBS2 | 0.028 | 2.45 | 7.5% | 0.014 | M1 = 6 |
| NM_152776A | IOH21708 | MGC40579-A | 0.035 | 3.22 | 7.5% | 0.014 | M1 = 6 |
| NM_152789 | IOH21683 | MGC40405 | 0.016 | 2.06 | 11.8% | 0.015 | M3 = 10 |
| NM_153229 | IOH40698 | TMEM92 | 0.025 | 1.80 | 12.9% | 0.009 | M3 = 11 |
| NM_153641A | IOH12153 | PANK2-A | 0.023 | 2.79 | 6.5% | 0.029 | M1 = 5 |
| NM_153702A | IOH21427 | ELMOD2-A | 0.047 | 2.79 | 9.7% | 0.048 | M3 = 8 |
| NM_170693A | IOH14023 | SGK2-A | 0.014 | 2.25 | 10.8% | 0.028 | M3 = 9 |
| NM_173192A | IOH21409 | KCNIP2-A | 0.028 | 2.36 | 7.5% | 0.014 | M1 = 6 |
| NM_174903A | IOH22522 | unknown-A | 0.044 | 2.24 | 12.9% | 0.009 | M3 = 11 |
| NM_174942A | IOH26291 | GAS2L3-A | 0.009 | 3.58 | 14.0% | 0.005 | M3 = 12 |
| NM_177938 | IOH40630 | PH-4 | 0.023 | 2.37 | 9.7% | 0.048 | M3 = 8 |
| NM_178543 | IOH26296 | ENPP7 | 0.021 | 1.80 | 9.7% | 0.048 | M3 = 8 |
| NM_201432A | IOH40915 | GAS7-A | 0.049 | 2.72 | 8.6% | 0.007 | M1 = 7 |
| NM_203284A | IOH39978 | RBPSUH-A | 0.040 | 4.31 | 11.8% | 0.015 | M3 = 10 |

TABLE 4
Target antigens that were downregulated in patients diagnosed with
lung adenocarcinoma compared to normal individuals.

| | | | t-test | RATIO | BEST | BEST | |
|---|---|---|---|---|---|---|---|
| 141 Down in LAC | | | p-value | 3-NO + | M-Stat | M-Stat | BEST |
| UI1 | UI2 | SYMBOL | C, N, 1, 3 | 3-CA | Prev | p-value | M-Stat |
| BC000108A | IOH4735 | WWP2-A | 0.014 | 2.60 | 8.7% | 0.087 | M3 = 7 |
| BC000479A | IOH3692 | AKT1-A | 0.031 | 2.18 | 6.5% | 0.030 | M1 = 5 |
| BC001052 | IOH3752 | RECQL | 0.023 | 1.77 | 10.9% | 0.030 | M3 = 9 |
| BC007015 | IOH29312 | CCNE2 | 0.047 | 3.94 | 5.4% | 0.062 | M1 = 4 |
| BC007566 | IOH6849 | M6PRBP1 | 0.046 | 3.11 | 6.5% | 0.030 | M1 = 5 |
| BC007957A | IOH6766 | C20orf116-A | 0.007 | 1.97 | 8.7% | 0.033 | M2 = 7 |
| BC008217A | IOH3169 | HNRPLL-A | 0.032 | 1.80 | 6.5% | 0.030 | M1 = 5 |
| BC008253 | IOH3366 | C8orf43 | 0.017 | 2.03 | 14.1% | 0.005 | M3 = 12 |
| BC008623A | IOH3309 | ROBO3-A | 0.012 | 1.79 | 12.0% | 0.017 | M3 = 10 |
| BC008730A | IOH5942 | HK1-A | 0.030 | 3.17 | 9.8% | 0.052 | M3 = 8 |
| BC009047 | IOH9807 | PDE9A | 0.012 | 2.18 | 7.6% | 0.061 | M2 = 6 |
| BC009047A | IOH9807 | PDE9A-A | 0.029 | 2.32 | 6.5% | 0.108 | M2 = 5 |
| BC009289 | IOH12925 | ACSBG1 | 0.037 | 1.81 | 10.9% | 0.030 | M3 = 9 |
| BC011989A | IOH11771 | ESM1-A | 0.018 | 2.44 | 8.7% | 0.033 | M2 = 7 |
| BC012021 | IOH10818 | RNF125 | 0.024 | 1.89 | 4.3% | 0.125 | M1 = 3 |
| BC012094A | IOH13126 | RABGAP1L-A | 0.028 | 2.12 | 5.4% | 0.187 | M2 = 4 |
| BC012616A | IOH13744 | BHMT-A | 0.031 | 4.21 | 9.8% | 0.018 | M2 = 8 |
| BC012814A | IOH13051 | ZFPL1-A | 0.041 | 2.02 | 7.6% | 0.015 | M1 = 6 |
| BC014131A | IOH12938 | RPUSD4-A | 0.031 | 2.51 | 7.6% | 0.061 | M2 = 6 |
| BC014244 | IOH12917 | RTN2 | 0.020 | 2.37 | 7.6% | 0.061 | M2 = 6 |
| BC014435 | IOH14630 | HCK | 0.008 | 2.06 | 6.5% | 0.030 | M1 = 5 |
| BC014959A | IOH13467 | RYBP-A | 0.041 | 2.86 | 5.4% | 0.062 | M1 = 4 |
| BC014969A | IOH12203 | ATF6-A | 0.006 | 2.22 | 8.7% | 0.007 | M1 = 7 |
| BC015614A | IOH14601 | RGS20-A | 0.006 | 3.23 | 13.0% | 0.000 | M1 = 11 |
| BC015877 | IOH13013 | CDH19 | 0.005 | 1.77 | 9.8% | 0.018 | M2 = 8 |
| BC016652 | NotAvailable | BMX | 0.044 | 3.01 | 9.8% | 0.052 | M3 = 8 |
| BC019823 | IOH11439 | C20orf161 | 0.005 | 2.00 | 7.6% | 0.015 | M1 = 6 |
| BC020203A | IOH14766 | GDPD5-A | 0.037 | 4.26 | 6.5% | 0.030 | M1 = 5 |
| BC020729A | IOH12978 | CCNE2-A | 0.010 | 1.85 | 8.7% | 0.033 | M2 = 7 |
| BC020981 | IOH10388 | GTF3C2 | 0.019 | 1.81 | 13.0% | 0.003 | M2 = 11 |
| BC022248 | IOH12822 | DNAJB14 | 0.031 | 1.83 | 6.5% | 0.030 | M1 = 5 |
| BC022429 | IOH23036 | LOC90624 | 0.033 | 2.06 | 5.4% | 0.187 | M2 = 4 |
| BC022429A | IOH23036 | LOC90624-A | 0.008 | 2.52 | 6.5% | 0.030 | M1 = 5 |
| BC024001A | IOH11161 | LRRC20-A | 0.012 | 4.27 | 10.9% | 0.002 | M1 = 9 |
| BC026100A | IOH11308 | TTTY8-A | 0.027 | 2.72 | 10.9% | 0.010 | M2 = 9 |
| BC029529 | IOH27222 | TUBB2 | 0.005 | 1.80 | 9.8% | 0.003 | M1 = 8 |
| BC029580 | IOH21597 | ENSG00000180586 | 0.006 | 1.85 | 6.5% | 0.030 | M1 = 5 |
| BC030808A | IOH23056 | ZFYVE16-A | 0.033 | 2.10 | 4.3% | 0.125 | M1 = 3 |
| BC031228A | IOH21599 | ENSG00000180807-A | 0.009 | 1.75 | 10.9% | 0.010 | M2 = 9 |
| BC031695A | IOH21499 | RP11-529I10.4-A | 0.032 | 3.61 | 8.7% | 0.007 | M1 = 7 |
| BC032656A | IOH21979 | TCF7L2-A | 0.034 | 2.25 | 7.6% | 0.015 | M1 = 6 |
| BC032852A | IOH27153 | MAGEB4-A | 0.035 | 2.40 | 8.7% | 0.087 | M3 = 7 |
| BC033178A | IOH23236 | IGHG3-A | 0.020 | 2.64 | 12.0% | 0.017 | M3 = 10 |
| BC033622A | IOH21694 | IMPDH1-A | 0.041 | 4.36 | 7.6% | 0.015 | M1 = 6 |
| BC033790 | IOH21793 | C1orf92 | 0.034 | 1.96 | 6.5% | 0.030 | M1 = 5 |
| BC034222A | IOH21594 | HRLP5-A | 0.009 | 3.25 | 13.0% | 0.010 | M3 = 11 |
| BC035636 | IOH27581 | APBB1IP | 0.043 | 1.94 | 9.8% | 0.052 | M3 = 8 |
| BC038105 | IOH27173 | MPP7 | 0.020 | 1.97 | 8.7% | 0.087 | M3 = 7 |
| BC040280A | IOH27452 | MGC34732-A | 0.032 | 4.36 | 13.0% | 0.010 | M3 = 11 |
| BC040844A | IOH26310 | SYNCRIP-A | 0.038 | 2.37 | 6.5% | 0.030 | M1 = 5 |
| BC041031 | IOH28000 | MSH5 | 0.025 | 2.49 | 6.5% | 0.030 | M1 = 5 |
| BC045563 | IOH26744 | FLJ30934 | 0.035 | 1.91 | 7.6% | 0.015 | M1 = 6 |
| BC046449A | IOH28670 | MGC35402-A | 0.048 | 2.24 | 4.3% | 0.125 | M1 = 3 |
| BC047480A | IOH26523 | DAZ4-A | 0.023 | 2.03 | 9.8% | 0.052 | M3 = 8 |
| BC050563A | IOH26951 | LOC202051-A | 0.052 | 2.31 | 7.6% | 0.143 | M3 = 6 |
| BC055314A | IOH29446 | EMG1-A | 0.032 | 2.96 | 5.4% | 0.062 | M1 = 4 |
| BC057774A | IOH29168 | RG9MTD3-A | 0.043 | 3.73 | 6.5% | 0.030 | M1 = 5 |
| BC068537 | IOH40089 | SPTLC1 | 0.029 | 2.53 | 5.4% | 0.062 | M1 = 4 |
| NM_000045A | IOH14233 | ARG1-A | 0.012 | 2.23 | 7.6% | 0.015 | M1 = 6 |
| NM_000326 | IOH5232 | RLBP1 | 0.040 | 1.78 | 9.8% | 0.018 | M2 = 8 |
| NM_001154 | IOH5099 | ANXA5 | 0.038 | 1.83 | 13.0% | 0.010 | M3 = 11 |
| NM_001277 | IOH21323 | CHKA | 0.009 | 2.07 | 10.9% | 0.002 | M1 = 9 |
| NM_001312A | IOH3437 | CRIP2-A | 0.026 | 2.64 | 9.8% | 0.052 | M3 = 8 |
| NM_001721 | IOH11645 | BMX | 0.020 | 3.47 | 10.9% | 0.030 | M3 = 9 |
| NM_001722A | IOH4103 | POLR3D-A | 0.012 | 2.58 | 8.7% | 0.007 | M1 = 7 |
| NM_001760 | IOH14467 | CCND3 | 0.028 | 2.04 | 16.3% | 0.001 | M3 = 14 |
| NM_002521 | IOH22623 | NPPB | 0.047 | 2.39 | 4.3% | 0.125 | M1 = 3 |
| NM_002572 | IOH3561 | PAFAH1B2 | 0.053 | 1.76 | 6.5% | 0.108 | M2 = 5 |
| NM_003104 | IOH14671 | SORD | 0.033 | 2.12 | 9.8% | 0.003 | M1 = 8 |
| NM_003277A | IOH3589 | CLDN5-A | 0.025 | 1.88 | 9.8% | 0.052 | M3 = 8 |
| NM_003362 | IOH11675 | UNG | 0.051 | 1.99 | 8.7% | 0.007 | M1 = 7 |
| NM_003372A | IOH26968 | VBP1-A | 0.047 | 2.02 | 9.8% | 0.052 | M3 = 8 |
| NM_003390A | NotAvailable | WEE1-A | 0.030 | 1.97 | 13.0% | 0.010 | M3 = 11 |
| NM_003617 | IOH23200 | RGS5 | 0.032 | 3.20 | 8.7% | 0.033 | M2 = 7 |
| NM_003668A | IOH28010 | MAPKAPK5-A | 0.019 | 2.94 | 8.7% | 0.007 | M1 = 7 |
| NM_004240A | IOH12722 | TRIP10-A | 0.044 | 4.06 | 6.5% | 0.030 | M1 = 5 |
| NM_004708A | IOH11821 | PDCD5-A | 0.038 | 2.99 | 5.4% | 0.062 | M1 = 4 |
| NM_004853 | IOH9940 | STX8 | 0.017 | 2.07 | 12.0% | 0.017 | M3 = 10 |
| NM_005842A | IOH13536 | SPRY2-A | 0.027 | 3.64 | 13.0% | 0.010 | M3 = 11 |
| NM_005851A | IOH5446 | CDK2AP2-A | 0.044 | 2.60 | 7.6% | 0.061 | M2 = 6 |
| NM_006112A | IOH5219 | PPIE-A | 0.051 | 3.53 | 9.8% | 0.052 | M3 = 8 |
| NM_006212A | IOH34755 | PFKFB2-A | 0.006 | 3.31 | 12.0% | 0.005 | M2 = 10 |
| NM_006374 | IOH6735 | STK25 | 0.016 | 1.87 | 8.7% | 0.033 | M2 = 7 |
| NM_006401 | IOH3664 | ANP32B | 0.024 | 4.38 | 7.6% | 0.015 | M1 = 6 |
| NM_006555 | IOH5843 | YKT6 | 0.048 | 2.62 | 14.1% | 0.001 | M2 = 12 |
| NM_006555A | IOH5843 | YKT6-A | 0.047 | 5.35 | 8.7% | 0.033 | M2 = 7 |
| NM_006695 | IOH5798 | RPIP8 | 0.017 | 1.76 | 13.0% | 0.010 | M3 = 11 |
| NM_006870A | IOH12247 | DSTN-A | 0.003 | 1.98 | 14.1% | 0.005 | M3 = 12 |
| NM_007202 | IOH10972 | AKAP10 | 0.019 | 5.64 | 12.0% | 0.017 | M3 = 10 |
| NM_007240A | IOH6325 | DUSP12-A | 0.034 | 2.40 | 9.8% | 0.052 | M3 = 8 |
| NM_007373A | IOH26711 | SHOC2-A | 0.037 | 1.81 | 8.7% | 0.033 | M2 = 7 |
| NM_012179A | IOH7010 | FBXO7-A | 0.037 | 1.84 | 9.8% | 0.052 | M3 = 8 |
| NM_014284 | IOH11121 | NCDN | 0.006 | 5.25 | 6.5% | 0.030 | M1 = 5 |
| NM_014551A | IOH3660 | hCAP-H2-A | 0.018 | 1.82 | 9.8% | 0.018 | M2 = 8 |
| NM_015201A | IOH14400 | BOP1-A | 0.018 | 2.03 | 7.6% | 0.061 | M2 = 6 |
| NM_016185 | IOH4078 | HN1 | 0.009 | 1.76 | 6.5% | 0.030 | M1 = 5 |
| NM_016234A | IOH6123 | ACSL5-A | 0.032 | 1.94 | 6.5% | 0.030 | M1 = 5 |
| NM_016355A | IOH39973 | DDX47-A | 0.037 | 1.80 | 4.3% | 0.125 | M1 = 3 |
| NM_016440A | NotAvailable | VRK3-A | 0.037 | 3.19 | 6.5% | 0.030 | M1 = 5 |
| NM_016561A | IOH4880 | BFAR-A | 0.043 | 1.94 | 9.8% | 0.052 | M3 = 8 |
| NM_016940 | IOH12821 | C21orf6 | 0.030 | 1.81 | 4.3% | 0.125 | M1 = 3 |
| NM_017612 | IOH11180 | ZCCHC8 | 0.022 | 2.20 | 6.5% | 0.030 | M1 = 5 |
| NM_019021A | IOH3368 | FLJ20010-A | 0.034 | 1.84 | 12.0% | 0.005 | M2 = 10 |
| NM_019612 | IOH40077 | IRGC1 | 0.005 | 1.85 | 10.9% | 0.030 | M3 = 9 |
| NM_020247A | IOH40578 | CABC1-A | 0.027 | 1.80 | 13.0% | 0.010 | M3 = 11 |
| NM_020548 | IOH39853 | DBI | 0.050 | 2.51 | 9.8% | 0.052 | M3 = 8 |
| NM_020677A | IOH5739 | HSCARG-A | 0.045 | 2.61 | 8.7% | 0.007 | M1 = 7 |
| NM_021972A | IOH13586 | SPHK1-A | 0.019 | 2.36 | 13.0% | 0.003 | M2 = 11 |
| NM_022110A | IOH10458 | FKBPL-A | 0.018 | 5.63 | 7.6% | 0.015 | M1 = 6 |
| NM_022823A | IOH21980 | FNDC4-A | 0.038 | 2.15 | 7.6% | 0.015 | M1 = 6 |
| NM_023935A | IOH4217 | C20orf116-A | 0.049 | 1.80 | 6.5% | 0.108 | M2 = 5 |
| NM_024051 | IOH4274 | C7orf24 | 0.015 | 3.82 | 6.5% | 0.030 | M1 = 5 |
| NM_024578A | IOH23128 | FLJ22709-A | 0.016 | 2.90 | 9.8% | 0.003 | M1 = 8 |
| NM_024805 | IOH13501 | C18orf22 | 0.008 | 4.28 | 16.3% | 0.001 | M3 = 14 |
| NM_025136A | IOH6524 | OPA3-A | 0.053 | 2.21 | 12.0% | 0.017 | M3 = 10 |
| NM_030768A | IOH12865 | ILKAP-A | 0.043 | 8.87 | 6.5% | 0.030 | M1 = 5 |
| NM_032023 | IOH21970 | RASSF4 | 0.040 | 1.82 | 8.7% | 0.087 | M3 = 7 |
| NM_032214A | IOH26309 | SLA2-A | 0.023 | 3.20 | 13.0% | 0.010 | M3 = 11 |
| NM_032318A | IOH6525 | HIATL2-A | 0.013 | 1.77 | 9.8% | 0.052 | M3 = 8 |
| NM_032341 | IOH7525 | DDI2 | 0.031 | 3.38 | 9.8% | 0.003 | M1 = 8 |
| NM_032439 | IOH40856 | PHYHIPL | 0.044 | 3.36 | 7.6% | 0.015 | M1 = 6 |
| NM_032728A | IOH40606 | PPAPDC3-A | 0.006 | 1.78 | 8.7% | 0.033 | M2 = 7 |
| NM_033397 | IOH27489 | KIAA1754 | 0.025 | 1.99 | 4.3% | 0.125 | M1 = 3 |
| NM_053046A | IOH27186 | EGLN2-A | 0.007 | 2.44 | 6.5% | 0.030 | M1 = 5 |
| NM_080423 | IOH23012 | PTPN2 | 0.033 | 2.78 | 6.5% | 0.030 | M1 = 5 |
| NM_080719 | IOH14144 | MGC4473 | 0.033 | 2.48 | 9.8% | 0.018 | M2 = 8 |
| NM_133480 | IOH13139 | TADA3L | 0.003 | 2.88 | 8.7% | 0.007 | M1 = 7 |
| NM_138785A | IOH12456 | C6orf72-A | 0.037 | 2.01 | 8.7% | 0.087 | M3 = 7 |
| NM_144638A | IOH10667 | TMEM42-A | 0.037 | 2.11 | 13.0% | 0.010 | M3 = 11 |
| NM_145063 | IOH13839 | C6orf130 | 0.008 | 1.80 | 13.0% | 0.010 | M3 = 11 |
| NM_145265A | IOH10426 | LOC133957-A | 0.014 | 2.62 | 7.6% | 0.015 | M1 = 6 |
| NM_145280 | IOH21779 | LOC151194 | 0.015 | 2.66 | 7.6% | 0.015 | M1 = 6 |
| NM_152653 | IOH13176 | UBE2E2 | 0.009 | 1.80 | 10.9% | 0.010 | M2 = 9 |
| NM_152682 | IOH10053 | RWDD4A | 0.039 | 3.51 | 8.7% | 0.087 | M3 = 7 |
| NM_152732 | IOH22555 | C6orf206 | 0.015 | 4.97 | 12.0% | 0.001 | M1 = 10 |
| NM_178585 | IOH12602 | WDFY3 | 0.039 | 1.77 | 6.5% | 0.030 | M1 = 5 |
| NM_181795 | IOH27983 | PKIB | 0.010 | 2.46 | 12.0% | 0.017 | M3 = 10 |
| NM_182563 | IOH25780 | MGC21830 | 0.012 | 2.42 | 12.0% | 0.005 | M2 = 10 |
| NM_199326 | IOH3323 | PPP2R3B | 0.048 | 6.51 | 9.8% | 0.052 | M3 = 8 |
| NM_199328 | IOH12281 | CLDN8 | 0.006 | 2.15 | 7.6% | 0.015 | M1 = 6 |
| NM_199415A | IOH26936 | UBOX5-A | 0.046 | 2.60 | 6.5% | 0.030 | M1 = 5 |

Table 5 list target antigens associated with IgG that are bound more often by antibodies from sera from lung adenocarcinoma individuals than by antibodies from healthy individuals. Table 5 identifies antigens according to Genbank ID number for the nucleotide sequence that encodes the antigens. It is understood that an antigen of Table 5 refers to a protein or fragments thereof that is encoded by the nucleotide sequence associated with the nucleotide ID number.

TABLE 5
IgG target antigens bound more often by antibodies from
sera from lung adenocarcinoma individuals than by antibodies
from healthy individuals.

| | Acc | Symbol |
|---|---|---|
| 1 | BC000084 | FLJ10357 |
| 2 | BC000120 | GTF2F1 |
| 3 | BC000733 | EIF3S4 |
| 4 | BC000807 | ZNF160 |
| 5 | BC001120 | LGALS3 |
| 6 | BC001709 | CDC2L2 |
| 7 | BC002559 | YTHDF2 |
| 8 | BC002880 | CARS |
| 9 | BC003132 | NUDC |
| 10 | BC006222 | C14orf131 |
| 11 | BC007320 | ANXA10 |
| 12 | BC007363 | BCKDK |
| 13 | BC008058 | PRKCZ |
| 14 | BC009623 | NPM1 |
| 15 | BC010356 | WAC |
| 16 | BC010632 | FAM29A |
| 17 | BC011379 | C15orf39 |
| 18 | BC011454 | AMOTL2 |
| 19 | BC012289 | KIAA0515 |
| 20 | BC012997 | SULF1 |
| 21 | BC014001 | UBXD8 |
| 22 | BC014218 | |
| 23 | BC014959 | RYBP |
| 24 | BC014991 | MPG |
| 25 | BC015417 | RPL24 |
| 26 | BC016380 | IGKC |
| 27 | BC017070 | CLUAP1 |
| 28 | BC018302 | TRMT1 |
| 29 | BC018929 | PHLDA1 |
| 30 | BC021263 | RAB24 |
| 31 | BC021282 | ZNF444 |
| 32 | BC023505 | ECM1 |
| 33 | BC027607 | ZC3H14 |
| 34 | BC027609 | SPERT |
| 35 | BC028039 | MGC39900 |
| 36 | BC028040 | CNP |
| 37 | BC028301 | C18orf56 |
| 38 | BC029541 | LETM2 |
| 39 | BC030808 | ZFYVE16 |
| 40 | BC031228 | |
| 41 | BC031964 | GLUL |
| 42 | BC032449 | PALM |
| 43 | BC032499 | GBGT1 |
| 44 | BC033153 | SCARA5 |
| 45 | BC033629 | C20orf77 |
| 46 | BC034376 | AMPH |
| 47 | BC035143 | TIGD1 |
| 48 | BC035601 | WWC3 |
| 49 | BC036075 | DNAJB4 |
| 50 | BC037906 | C4orf19 |
| 51 | BC038838 | LOC51334 |
| 52 | BC039306 | PALM2-AKAP2 |
| 53 | BC042482 | KCTD7 |
| 54 | BC048125 | GALK2 |
| 55 | BC051762 | C20orf96 |
| 56 | BC051885 | C14orf106 |
| 57 | BC052303 | ARHGAP4 |
| 58 | BC052601 | MRPL10 |
| 59 | BC052639 | MRPL43 |
| 60 | BC053373 | MRPL43 |
| 61 | BC053656 | EDIL3 |
| 62 | BC053866 | EDN3 |
| 63 | BC056409 | CROP |
| 64 | BC058843 | ZBTB8OS |
| 65 | NM_000137 | FAH |
| 66 | NM_000449 | RFX5 |
| 67 | NM_001094 | ACCN1 |
| 68 | NM_001203 | BMPR1B |
| 69 | NM_001280 | CIRBP |
| 70 | NM_001612 | ACRV1 |
| 71 | NM_001997 | FAU |
| 72 | NM_002046 | GAPDH |
| 73 | NM_002419 | MAP3K11 |
| 74 | NM_002491 | NDUFB3 |
| 75 | NM_002744 | PRKCZ |
| 76 | NM_002944 | ROS1 |
| 77 | NM_002945 | RPA1 |
| 78 | NM_003885 | CDK5R1 |
| 79 | NM_003992 | CLK3 |
| 80 | NM_004055 | CAPN5 |
| 81 | NM_004281 | BAG3 |
| 82 | NM_004304 | ALK |
| 83 | NM_004384 | CSNK1G3 |
| 84 | NM_004656 | BAP1 |
| 85 | NM_004708 | PDCD5 |
| 86 | NM_004846 | EIF4E2 |
| 87 | NM_004952 | EFNA3 |
| 88 | NM_005002 | NDUFA9 |
| 89 | NM_005011 | NRF1 |
| 90 | NM_005252 | FOS |
| 91 | NM_005299 | GPR31 |
| 92 | NM_005313 | PDIA3 |
| 93 | NM_005406 | ROCK1 |
| 94 | NM_005435 | ARHGEF5 |
| 95 | NM_006117 | PECI |
| 96 | NM_006166 | NFYB |
| 97 | NM_006428 | MRPL28 |
| 98 | NM_006609 | MAP3K2 |
| 99 | NM_006857 | RY1 |
| 100 | NM_012101 | TRIM29 |
| 101 | NM_012424 | RPS6KC1 |
| 102 | NM_014044 | UNC50 |
| 103 | NM_015640 | SERBP1 |
| 104 | NM_016034 | MRPS2 |
| 105 | NM_016052 | CGI-115 |
| 106 | NM_016304 | C15orf15 |
| 107 | NM_018466 | GLT28D1 |
| 108 | NM_020175 | DUS3L |
| 109 | NM_020317 | C1orf63 |
| 110 | NM_020347 | LZTFL1 |
| 111 | NM_020444 | KIAA1191 |
| 112 | NM_020954 | KIAA1618 |
| 113 | NM_021822 | APOBEC3F |
| 114 | NM_021945 | C6orf85 |
| 115 | NM_024893 | C20orf39 |
| 116 | NM_024946 | NIP30 |
| 117 | NM_025136 | OPA3 |
| 118 | NM_030645 | SH3BP5L |
| 119 | NM_031417 | MARK4 |
| 120 | NM_032693 | |
| 121 | NM_052845 | MMAB |
| 122 | NM_052848 | CCDC97 |
| 123 | NM_130809 | LOC133619 |
| 124 | NM_133494 | NEK7 |
| 125 | NM_144710 | |
| 126 | NM_152474 | C19orf18 |
| 127 | NM_152576 | |
| 128 | NM_152723 | CCDC89 |
| 129 | NM_152770 | C4orf22 |
| 130 | NM_153043 | |
| 131 | NM_153215 | IFRD2 |
| 132 | NM_173494 | CXorf41 |
| 133 | NM_198395 | G3BP |
| 134 | NM_203454 | APOBEC4 |

Table 6 list target antigens associated with IgA that are bound more often by antibodies from sera from lung adenocarcinoma individuals than by antibodies from healthy individuals. Table 6 identifies antigens according to Genbank ID number for the nucleotide sequence that encodes the antigens. It is understood that an antigen of Table 6 refers to a protein or fragments thereof that is encoded by the nucleotide sequence associated with the nucleotide ID number.

TABLE 6
IgA target antigens bound more often by antibodies
from sera from lung adenocarcinoma individuals
than by antibodies from healthy individuals.

| | Acc | Symbol |
|---|---|---|
| 1 | BC000288A | CNBP-A |
| 2 | BC002755A | MKNK1-A |
| 3 | BC003065A | CDK2-A |
| 4 | BC003666A | NADSYN1-A |
| 5 | BC005153A | RPH3AL-A |
| 6 | BC005378A | C4BPB-A |
| 7 | BC006318A | EPB49-A |
| 8 | BC007028A | ELA3B-A |
| 9 | BC007315A | CNOT7-A |
| 10 | BC007382A | RAB7B-A |
| 11 | BC007411A | DIAPH1-A |
| 12 | BC008077A | SRPR-A |
| 13 | BC009010A | C6orf142-A |
| 14 | BC009894A | PAPSS2-A |
| 15 | BC010152A | STOML2-A |
| 16 | BC010356A | WAC-A |
| 17 | BC010935A | CSN3-A |
| 18 | BC011454A | AMOTL2-A |
| 19 | BC012105A | NVL-A |
| 20 | BC012289A | KIAA0515-A |
| 21 | BC013073A | C1orf37-A |
| 22 | BC013966A | FAM64A-A |
| 23 | BC014298A | PMS2L3-A |
| 24 | BC014299A | C14orf93-A |
| 25 | BC014435A | HCK-A |
| 26 | BC015239A | ZBTB8-A |
| 27 | BC016645A | PSAT1-A |
| 28 | BC016842A | LSM14A-A |
| 29 | BC017570A | C9orf78-A |
| 30 | BC017724A | FIP1L1-A |
| 31 | BC017943A | PPP1R1C-A |
| 32 | BC018302A | TRMT1-A |
| 33 | BC018929A | PHLDA1-A |
| 34 | BC019358A | TTYH1-A |
| 35 | BC019598A | ZMAT4-A |
| 36 | BC020221A | STAC-A |
| 37 | BC020555A | SERBP1-A |
| 38 | BC020651A | MRPL35-A |
| 39 | BC021263A | RAB24-A |
| 40 | BC024289A | IGHG1-A |
| 41 | BC025243A | CCDC8-A |
| 42 | BC025996A | LOC441046-A |
| 43 | BC029529A | TUBB2C-A |
| 44 | BC029566A | DMRTB1-A |
| 45 | BC030586A | STAM-A |
| 46 | BC031300A | C21orf2-A |
| 47 | BC032452A | IGL@-A |
| 48 | BC032485A | AIFL-A |
| 49 | BC032499A | GBGT1-A |
| 50 | BC033034A | DIXDC1-A |
| 51 | BC033621A | PUS7L-A |
| 52 | BC034376A | AMPH-A |
| 53 | BC034401A | |
| 54 | BC034528A | SERPINB8-A |
| 55 | BC036767A | RIBC1-A |
| 56 | BC036910A | LOC388882-A |
| 57 | BC038105A | MPP7-A |
| 58 | BC043581A | KRT36-A |
| 59 | BC043619A | MBD3-A |
| 60 | BC047722A | MGC52110-A |
| 61 | BC051366A | ZNF75A-A |
| 62 | BC052303A | ARHGAP4-A |
| 63 | BC053545A | TPM1-A |
| 64 | BC053600A | TMCO4-A |
| 65 | BC053866A | EDN3-A |
| 66 | BC056402A | LOC144097-A |
| 67 | BC059405A | TLE4-A |
| 68 | BC060773A | SOX5-A |
| 69 | BC060813A | AMMECR1-A |
| 70 | BC065525A | ADD2-A |
| 71 | BC066987A | DPY19L2P1-A |
| 72 | BC067735A | DKFZp761B107-A |
| 73 | BC067773A | FLJ11021-A |
| 74 | NM_000215A | JAK3-A |
| 75 | NM_000559A | HBG1-A |
| 76 | NM_000714A | TSPO-A |
| 77 | NM_001002018A | HCFC1R1-A |
| 78 | NM_001094A | ACCN1-A |
| 79 | NM_001167A | BIRC4-A |
| 80 | NM_001544A | ICAM4-A |
| 81 | NM_001612A | ACRV1-A |
| 82 | NM_001663A | ARF6-A |
| 83 | NM_001833A | CLTA-A |
| 84 | NM_001935A | DPP4-A |
| 85 | NM_001950A | E2F4-A |
| 86 | NM_002031A | FRK-A |
| 87 | NM_002419A | MAP3K11-A |
| 88 | NM_002431A | MNAT1-A |
| 89 | NM_002436A | MPP1-A |
| 90 | NM_002734A | PRKAR1A-A |
| 91 | NM_002749A | MAPK7-A |
| 92 | NM_003192A | TBCC-A |
| 93 | NM_003321A | TUFM-A |
| 94 | NM_004461A | FARSLA-A |
| 95 | NM_004772A | C5orf13-A |
| 96 | NM_004782A | SNAP29-A |
| 97 | NM_004783A | TAOK2-A |
| 98 | NM_004952A | EFNA3-A |
| 99 | NM_004997A | MYBPH-A |
| 100 | NM_005038A | PPID-A |
| 101 | NM_005160A | ADRBK2-A |
| 102 | NM_005550A | KIFC3-A |
| 103 | NM_005592A | MUSK-A |
| 104 | NM_005720A | ARPC1B-A |
| 105 | NM_005770A | |
| 106 | NM_006205A | PDE6H-A |
| 107 | NM_006293A | TYRO3-A |
| 108 | NM_006573A | TNFSF13B-A |
| 109 | NM_006695A | RPIP8-A |
| 110 | NM_006978A | RNF113A-A |
| 111 | NM_007107A | SSR3-A |
| 112 | NM_013974A | DDAH2-A |
| 113 | NM_013975A | LIG3-A |
| 114 | NM_014044A | UNC50-A |
| 115 | NM_014110A | PPP1R8-A |
| 116 | NM_014215A | INSRR-A |
| 117 | NM_014790A | JAKMIP2-A |
| 118 | NM_015138A | RTF1-A |
| 119 | NM_015246A | MGRN1-A |
| 120 | NM_015959A | CTNND1-A |
| 121 | NM_016034A | MRPS2-A |
| 122 | NM_016457A | PRKD2-A |
| 123 | NM_016836A | RBMS1-A |
| 124 | NM_018025A | GPATC1-A |
| 125 | NM_018047A | RBM22-A |
| 126 | NM_020175A | DUS3L-A |
| 127 | NM_020963A | MOV10-A |
| 128 | NM_022104A | C20orf67-A |
| 129 | NM_024041A | SCNM1-A |
| 130 | NM_024749A | VASH2-A |
| 131 | NM_024946A | NIP30-A |
| 132 | NM_025126A | RNF34-A |
| 133 | NM_025221A | KCNIP4-A |
| 134 | NM_032023A | RASSF4-A |
| 135 | NM_032168A | WDR75-A |
| 136 | NM_032332A | MAN2B1-A |
| 137 | NM_032929A | |
| 138 | NM_033019A | |
| 139 | NM_052845A | MMAB-A |
| 140 | NM_052848A | CCDC97-A |
| 141 | NM_054016A | FUSIP1-A |
| 142 | NM_080659A | C11orf52-A |
| 143 | NM_152318A | C12orf45-A |
| 144 | NM_152362A | TNFAIP8L1-A |
| 145 | NM_152638A | C12orf12-A |
| 146 | NM_152663A | RALGPS2-A |
| 147 | NM_152763A | C1orf62-A |
| 148 | NM_152786A | C9orf43-A |
| 149 | NM_153043A | |
| 150 | NM_153346A | CXorf20-A |
| 151 | NM_170676A | MEIS2-A |
| 152 | NM_173494A | CXorf41-A |
| 153 | NM_173565A | LOC222967-A |
| 154 | NM_173618A | CCDC95-A |
| 155 | NM_177924A | ASAH1-A |
| 156 | NM_178818A | CMTM4-A |
| 157 | NM_182691A | SRPK2-A |
| 158 | NM_199001A | MGC59937-A |

### Example 4

The general approach used in Immune Response Profiling data analysis employs a three-step process:

Single array analysis: For each protein on each array, a series of values is calculated including background subtracted signals, t_test and M-statistics

Group characterization: Signals for each individual protein across all samples from a given population are aligned for downstream analysis

Identify differences between treated and untreated sample populations: Utilizing M-statistics, proteins are identified for which the differential signals between two populations result in a significant p-value

A set of algorithms were used to compare groups and identify proteins which exhibit increased signal values in one group relative to another. M-Statistic values are reported, which are described below. In addition, a p-value is calculated for each protein across a comparison that represents the probability that there is no signal increase in one group compared to another.

### M-Statistics

This algorithm provides a count corresponding to the number of assays in one group for which a signal value for a specified protein is larger then the largest observed signal value for this protein in another group (smaller ellipse). Software can be used to subsequently calculate the number of arrays in a specified group with signals arising from this protein that are larger then the second largest signal in another group (larger ellipse), third largest etc., proceeding iteratively through the data set for all ProtoArray® proteins.

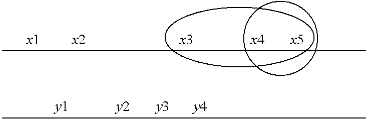

The M “I” order statistic for the group y of size n_{y} compared to group x of size nₓ is given by:

$${M}_{i , above , between}^{y} = {\sum }_{k = 1}^{{n}_{y}} {1}_{\{ {y}_{k} > {x}_{( i )} + between \}} {1}_{\{ {y}_{k} > above \}} \qquad ( 1 )$$

where x₍ᵢ₎ is the i^{th} largest value of the group x, and above and between are the calculation parameters.

The p-value is calculated as a probability of having an M value greater or equal then Mᵢ. The M statistic with the lowest p-value is selected and this Mₘₐₓ value and order is reported, as well as a corresponding p-value and prevalence estimate as described below.

Using a non-informative prior distribution for prevalence (i.e. assuming that the unknown prevalence of the marker is between 0 and 1) and acknowledging a binomial sampling scheme (i.e. that out of n arrays, the prevalence of the marker is given by p, one observes X arrays that are turned on), prevalence may be estimated as

$$E ( P ) = \frac{{M}_{max} + 1}{{n}_{y} + 2} . \qquad ( 2 )$$

### Quantile Normalization

Quantile normalization is a non-parametric procedure normalizing two or more one-channel datasets to a synthetic array. This method assumes that the distribution of signals is nearly the same in all samples. The largest signal for each array is replaced by a median value of the largest signals; the second largest signal is replaced by a median value of the second largest signals etc.

### Definitions of Statistical Terms

Hypothesis Testing: Two mutually exclusive hypotheses are given, one is typically called the null hypothesis and the other is typically called the alternative hypothesis. Data is then collected to test the viability of the null hypothesis, and this data is used to determine if the null hypothesis is rejected or not.

Rejection Rule: This is a statistical method in which the observed data either rejects the null hypothesis or fails to reject the null hypothesis. It is important to note that this Rule will never “accept the null or alternative hypothesis”; it is exclusively a rule to reject. There are four possible outcomes to this approach, based on the true nature of the null hypothesis, and what is decided by the Rejection Rule. The four outcomes can be shown as:

| | True Nature of H₀ | | |
|---|---|---|---|
| | | H₀ is True | H₀ is False |
| Decision by | Reject H₀ | Type I Error | Correct Decision |
| the Rejection | Fail to Reject | Correct | Type II Error |
| Rule | H₀ | Decision | |

Note that the true nature of Ho is never really known. The actual formula for the Rejection Rule varies from hypothesis test to hypothesis test depending on the type of data, and the set of assumptions being made.

Type I Error: Typically, the probability of a Type I error is denoted as a. In general this is considered the most serious type of error to make.

Type II Error: Typically the probability of a Type II error is denoted as A. Though this is also an error, it is usually controlled by attempting to minimize the probability of Type I Error.

Precision: In a statistical terminology, precision is defined as the probability of not making a Type I Error. This can be considered as the probability of a true positive. Hence this is denoted as 1-α.

Power: In a statistical terminology, power is defined as the probability of not making a Type II Error. This can be considered the probability of a true negative. Hence this is denoted as 1-β.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

One of ordinary skill in the art will appreciate that starting materials, reagents, purification methods, materials, substrates, device elements, analytical methods, assay methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

When a group of materials, compositions, components or compounds is disclosed herein, it is understood that all individual members of those groups and all subgroups thereof are disclosed separately. When a Markush group or other grouping is used herein, all individual members of the group and all combinations and subcombinations possible of the group are intended to be individually included in the disclosure. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. In the disclosure and the claims, “and/or” means additionally or alternatively. Moreover, any use of a term in the singular also encompasses plural forms.

All references cited herein are hereby incorporated by reference in their entirety to the extent that there is no inconsistency with the disclosure of this specification. Some references provided herein are incorporated by reference to provide details concerning sources of starting materials, additional starting materials, additional reagents, additional methods of synthesis, additional methods of analysis, additional biological materials, additional nucleic acids, chemically modified nucleic acids, additional cells, and additional uses of the invention. All headings used herein are for convenience only. All patents and publications mentioned in the specification are indicative of the levels of skill of those skilled in the art to which the invention pertains, and are herein incorporated by reference to the same extent as if each individual publication, patent or patent application was specifically and individually indicated to be incorporated by reference. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

What is claimed is:

1. A method of detecting one or more target antibodies in a test sample of an individual suspected of having lung adenocarcinoma comprising:
a) contacting the test sample from the individual with one or more target antigens each comprising an autoantigen of Table 1, Table 3, Table 4, Table 5 or Table 6 or a fragment thereof comprising an epitope recognized by a target antibody; and
b) detecting binding of one or more antibodies in the test sample to the one or more target antigens, thereby detecting the presence of the one or more target antibodies in the test sample.

2. The method of claim 1, wherein the one or more target antigens are immobilized on a solid support.

3. The method of claim 1, wherein the test sample is contacted with two or more target antigens of Table 1 or fragments thereof comprising an epitope.

4. The method of claim 1, wherein the test sample is contacted with twenty or more target antigens of Table 1 or fragments thereof comprising an epitope.

5. The method of claim 1, wherein the test sample is contacted with fifty or more target antigens of Table 1 or fragments thereof comprising an epitope.

6. The method of claim 1, wherein the test sample is contacted with seventy-five or more target antigens of Table 1 or fragments thereof comprising an epitope.

7. The method of claim 1, wherein the test sample is contacted with all of the target antigens of Table 1 or fragments thereof comprising an epitope.

8. The method of claim 1, wherein the test sample comprises cells, tissue, or a bodily fluid from the individual.

9. The method of claim 1, wherein the test sample comprises blood, serum, plasma, synovial fluid, cerebrospinal fluid, cell lysates or saliva from the individual.

10. The method of claim 1, further comprising detecting the amount of the one or more antibodies bound to the one or more target antigens in the test sample.

11. The method of claim 1, wherein binding of the one or more target antigens to one or more antibodies in the test sample is determined using an immunoassay.

12. The method of claim 1, wherein at least ten of the one or more target antigens are bound by the one or more antibodies from the test sample.

13. The method of claim 1, wherein at least twenty of the one or more target antigens are bound by the one or more antibodies from the test sample.

14. The method of claim 1, wherein at least fifty of the one or more target antigens are bound by the one or more antibodies from the test sample.

15. The method of claim 1, wherein said test sample is taken from the individual prior to pathology confirmed diagnosis of lung adenocarcinoma.

16. The method of claim 1, wherein said one or more target antigens comprise one or more antigens, or a fragment thereof comprising an epitope, selected from the group consisting of COQ3,LSM8, STAU, WDR27-A, WTAP-A, HEXIM1-A, and AHNAK-A.

17. A method of monitoring one or more target antibodies in test samples from an individual suspected as having lung adenocarcinoma comprising:
a) contacting a first test sample from the individual with a first set of one or more target antigens;
b) detecting binding of one or more antibodies in the test sample to the one or more target antigens, thereby detecting the presence of the one or more target antibodies in the first test sample;
c) contacting a second test sample from the individual with a second set of the one or more target antigens;
d) detecting binding of one or more antibodies in the test sample to the one or more target antigens, thereby detecting the presence of the one or more target antibodies in the second test sample; and
e) comparing the presence of the one or more antibodies from the first test sample with the presence of the one or more antibodies from the second test sample, wherein each of the one or more target antigens comprises an autoantigen of Table 1, Table 5 or Table 6 or fragments thereof comprising an epitope.

18. The method of claim 17, wherein the second test sample is taken from the individual at a later time than the first test sample.

19. The method of claim 17, further comprising the steps:
a) detecting the amount of the one or more antibodies bound to the one or more target antigens in the first test sample and the second test sample; and
b) comparing the amount of bound antibodies from the first test sample with the amount of bound antibodies from the second test sample.

20. A method of monitoring one or more target antibodies in test samples from an individual receiving treatment for lung adenocarcinoma comprising:
a) contacting a first test sample from an individual with a first set of one or more target antigens;
b) detecting binding of one or more antibodies in the first test sample to the one or more target antigens, thereby detecting the presence of the one or more target antibodies in the first test sample;
c) administering a treatment for lung adenocarcinoma to the individual;
d) after the administration of the treatment, contacting a second test sample from the individual with a second set of the one or more target antigens;
e) detecting binding of one or more antibodies in the second test sample to the one or more target antigens, thereby detecting the presence of the one or more target antibodies in the first second sample; and
f) comparing the presence of the one or more antibodies from the first sample with the presence of the one or more antibodies from the second sample, wherein each of the one or more target antigens comprises an autoantigen of Table 1, Table 5, Table 6 or fragments thereof comprising an epitope.

21. The method of claim 20, wherein the treatment comprises administering a drug to the individual.

22. The method of claim 20, wherein the treatment is continuous.

23. The method of claim 20, wherein the first test sample is taken from the individual prior to any treatment being administered to the individual.

24. The method of claim 20, further comprising contacting one or more subsequent test samples from the individual with an additional set of the one or more target antigens;
e) detecting binding of one or more antibodies in the one or more subsequent test samples to the one or more target antigens, thereby detecting the presence of the one or more target antibodies in the subsequent test samples; and
f) comparing the presence of the one or more antibodies from the subsequent test samples with the presence of one or more antibodies from the first and second sample, wherein each of the one or more target antigens comprises an autoantigen of Table 1, Table 5 or Table 6 or fragments thereof comprising an epitope.

25. The method of claim 20, further comprising:
a) detecting the amount of the one or more antibodies bound to the one or more target antigens in the first test sample and the second test sample; and
b) comparing the amount of bound antibodies from the first test sample with the amount of bound antibodies from the second test sample.

26. A kit for diagnosing lung adenocarcinoma comprising:
a) one or more target antigens each comprising an autoantigen of Table 1, Table 3, Table 4, Table 5 or Table 6 or a fragment thereof comprising an epitope; and
b) means for detecting binding of one or more molecules in a test sample to the one or more target antigens.

27. The kit of claim 26, wherein said one or more target antigens comprise one or more antigens, or a fragment thereof comprising an epitope, selected from the group consisting of COQ3,LSM8, STAU, WDR27-A, WTAP-A, HEXIM1-A, and AHNAK-A.

28. The kit of claim 26, further comprising a control antibody against one or more of the target antigens.

29. The kit of claim 26, wherein the kit comprises two or more target antigens each comprising an autoantigen of Table 1 or a fragment thereof comprising an epitope.

30. The kit of claim 26, wherein the kit comprises twenty or more target antigens each comprising an autoantigen of Table 1 or a fragment thereof comprising an epitope.

31. The kit of claim 26, wherein the kit comprises fifty or more target antigens each comprising an autoantigen of Table 1 or a fragment thereof comprising an epitope.

32. The kit of claim 26, wherein the one or more target antigens are immobilized on a solid support.

33. The kit of claim 26, wherein the one or more target antigens are part of a chip or high density protein array.

34. The kit of claim 26, wherein the one or more target antigens are immobilized on a plurality of solid supports.

35. The kit of claim 34, wherein the solid supports are flat surfaces or beads.

36. The kit of claim 26, wherein the kit comprises less than 1,000 polypeptides.

37. The kit of claim 26, wherein the kit comprises less than 100 polypeptides.

38. The kit of claim 26, wherein at least 75% of the polypeptides are autoantigens of Table 1 or a fragment thereof comprising an epitope.

39. The kit of claim 26, wherein at least 90% of the polypeptides are autoantigens of Table 1 or a fragment thereof comprising an epitope.

40. A mixture comprising:
a) one or more target antigens each comprising an autoantigen of Table 1, Table 3, Table 4, Table 5 or Table 6 or a fragment thereof comprising an epitope; and
b) a test sample from an individual suspected of having lung adenocarcinoma.

41. The mixture of claim 40, further comprising a control antibody against one or more of the target antigens.

42. The mixture of claim 40, further comprising two or more target antigens of Table 1 or fragments thereof comprising an epitope.

43. The mixture of claim 40, further comprising twenty or more target antigens of Table 1 or fragments thereof comprising an epitope.

44. The mixture of claim 40, further comprising fifty or more target antigens of Table 1 or fragments thereof comprising an epitope.

45. The mixture of claim 40, further comprising all of the target antigens of Table 1 or fragments thereof comprising an epitope.

46. The mixture of claim 40, wherein the test sample comprises cells, tissue, or a bodily fluid from the individual.

47. The mixture of claim 40, wherein one or more antibodies of the test sample are bound to one or more of the target antigens.

48. The mixture of claim 40, wherein at least 75% of the polypeptides are autoantigens of Table 1 or a fragment thereof comprising an epitope.

49. The mixture of claim 40, wherein at least 90% of the polypeptides are autoantigens of Table 1 or a fragment thereof comprising an epitope.

50. The mixture of claim 40, wherein the target antigens are immobilized on a solid support.

51. A method of detecting one or more upregulated target antibodies in a test sample of an individual suspected of having lung adenocarcinoma comprising:
a) contacting a first test sample from the individual with a first set of one or more target antigens;
b) contacting a second test sample from an healthy individual with a second set of the one or more target antigens;
c) detecting the amount of one or more antibodies bound to the one or more target antigens in the first test sample and the second test sample; and
e) comparing the amount of bound antibodies from the first test sample with the amount of bound antibodies from the second test, wherein each of the one or more target antigens comprises an autoantigen of Table 3 or fragments thereof comprising an epitope.

52. A method of detecting one or more downregulated target antibodies in a test sample of an individual suspected of having lung adenocarcinoma comprising:
a) contacting a first test sample from the individual with a first set of one or more target antigens;
b) contacting a second test sample from an healthy individual with a second set of the one or more target antigens;
c) detecting the amount of one or more antibodies bound to the one or more target antigens in the first test sample and the second test sample; and
e) comparing the amount of bound antibodies from the first test sample with the amount of bound antibodies from the second test, wherein each of the one or more target antigens comprises an autoantigen of Table 4 or fragments thereof comprising an epitope.
